(19) United States
(12) Patent Application Publication
(10) Pub. No.: US 2011/0152866 A1
(43) Pub. Date: Jun. 23, 2011

(54) Intraosseous injection system

(76) Inventor: Eric J. Knutson, Granite Bay, CA (US)

(21) Appl. No.: 12/653,941

(22) Filed: Dec. 19, 2009

(51) Int. Cl. A61B 17/00; A61B 17/56

(52) U.S. Cl. 606/80; 606/86 R

(57) ABSTRACT

Apparatus and method for injecting a fluid through perforated gingiva (24), through perforated cortical bone (28) covered by gingiva (24), and into cancellous bone (30). The method utilizes a bone bit (10) and a sealing injector (40). Bit (10) consists of a perforator (12) for forming a small diameter conduit (26) through cortical bone (28), and a larger diameter countersink (14) for forming a gingival access (22), and for forming a bony access seat (32). Injector (40) forms a seal with gingiva (24) about gingival access (22), or with the proximal bone (32). The seal is formed so that injector (40) remains in fluid communication with conduit (26). Once the seal is formed, injector (40) sealingly injects fluid into conduit (26) and into cancellous bone (30).

### BACKGROUND

1. Field of Invention

This invention relates to dental injections, specifically to intraosseous injections.

2. Description of Prior Art

Dental intraosseous injections are made by anesthetizing the gingiva over cortical bone prior to forming a hole in the cortical bone. A fluid is then injected through the cortical bone hole and into the spongy cancellous bone below. The fluid is generally a local anesthetic for anesthetising the teeth and tissues near the injection site. The primary advantage of intraosseous injections is the immediate and profound onset of anesthesia.

Intraosseous injections are typically used to reinforce ineffective inferior alveolar nerve blocks. They are also used to reinforce infiltrations, posterior superior alveolar injections, and other injections. Despite somewhat inconvenient, uncomfortable, or time-consuming pre-anesthesia methods generally used, a small number of clinicians use intraosseous anesthesia as a primary anesthesia in place of inferior alveolar nerve blocks.

Many devices have been improvised to facilitate intraosseous injections. Of those, very few are commercially available for clinical use.

Dillon (U.S. Pat. No. 5,173,050) shows a small diameter perforator that readily bores a small hole through the cortical bone. The perforator diameter is 0.43 mm, and it requires minimal work to bore the hole. After the hole is bored, the perforator is readily removed from the hole, as it may be rotated during removal. A needle having a closely-fitted diameter is inserted through the entire length of the cortical bone hole until the tip rests well into cancellous bone. The needle diameter is 27 gauge, or 0.4128 mm and 8 mm in length. A needle having a very slightly smaller diameter is selected in an attempt to facilitate insertability of the needle into the bony hole, balanced against providing a degree of resistance to fluid backflow leakage out the hole and into the mouth. Fluid is injected from the needle into the cancellous bone, and the needle is removed from the mouth. For reinjection, the user must relocate the small hole in the cortical bone, and reinsert the needle into the hole.

A first disadvantage is difficulty locating the small diameter hole, even when attempted immediately after the hole is bored. The difficulty locating the small hole increases with time. Reinjections are frequently needed during procedures. After 20-30 minutes has elapsed following perforation of the gingiva and cortical bone, gingival bleeding has generally stopped, and the gingival hole has partly closed, effectively making a small hole even smaller. Once the gingival hole is located, locating the orifice of the small bony hole under the gingiva can also be difficult. The gingival hole can slump out of alignment with the bony hole, and interfere with visually assessing the bony hole location. As such, the small diameter bony hole substantially covered by gingiva can be difficult to locate. Further, even when the needle is correctly aligned with the orifice of the bony hole, the gingiva can deflect the needle away from the orifice of the bony hole during attempted insertion. Yet further, the small diameter of the bony hole orifice presents an inconveniently sized target requiring exact needle placement for needle insertion. A hunt and peck approach is frequently required to locate the orifice of the small bony hole. As such, needle insertion into the orifice of the bony hole is inconvenient and can require excessive time.

A second disadvantage is the substantial difficulty inserting the needle into the small bony hole once the needle has been partly inserted. This is due to the use of a needle having a diameter that is closely-fitted to the hole. A needle having a substantially smaller diameter than the bony hole is avoided in order to avoid substantial fluid leakage backflowing around the needle and into the mouth. A needle having the same diameter or larger is avoided in order to minimize bony interferences to the insertion of the needle fully into and through the hole. As such, Dillon uses a closely-fitted needle. The needle tip tends to catch on the small irregularities that substantially interfere with sliding the needle along the walls of the hole.

A third disadvantage is that fluid tends to backflow past the needle and into the mouth. This leakage occurs because the needle has a slightly smaller diameter than the bony hole, and does not form a fluid-tight seal against the bony hole. If the user fails to insert the needle fully through the bony hole due to the interferences, such leakage can be substantial.

Meller (U.S. Pat. No. 6,247,928) shows a bit having a small-diameter solid perforator (anti-clog needle) having a cutting tip for use in a dental contra-angle handpiece. The perforator is approximately 27 gauge. A large-diameter hollow sleeve (boring needle) coaxially surrounds the perforator, and also has a cutting tip. The sleeve is 21 gauge. The perforator protrudes about 3 mm distally beyond the sleeve.

The proximal portion of the sleeve has a drill coupling located on the sleeve's outer surface for engaging and rotating the sleeve. The perforator has a mated coupling for detachably engaging the sleeve drill coupling. The perforator coupling extends distally from a handpiece drive shank that comprises the proximal portion of the bit.

In use, the perforator pilots a small diameter hole through the cortical bone. The sleeve advances with the perforator, and enlarges the bony hole on the same handpiece insertion stroke. The perforator deflects debris from the sleeve lumen. Both perforator and sleeve fully perforate through the cortical bone until sleeve tip rests in cancellous bone. The perforator is then withdrawn from the sleeve by forcefully pulling back on the handpiece, thereby separating the sleeve drill coupling from the mated perforator coupling. Once the perforator has been removed, the sleeve remains seated in the bony hole with the sleeve tip in cancellous bone. The sleeve and drill coupling also protrude several millimeters outwardly from the surface of the gingiva.

A needle having an OD that is somewhat closely-fitted to the ID of the sleeve is readily inserted entirely through the sleeve until the needle tip protrudes distally from the tip of the sleeve, and rests in the cancellous bone. There is no significant interference to needle insertion. However, the needle does not form a fluid-tight fit with the sleeve. Fluid is injected from the needle into the cancellous bone and then removed from the mouth. After injection, the sleeve remains inserted into the bony hole until completion of the procedure.

For reinjection, the user readily locates the orifice of the sleeve, and readily reinserts the needle into the sleeve. At the completion of the procedure, the sleeve is removed from the bony hole with a hemostat.

A first disadvantage is that perforating the cortical bone is difficult with the large diameter sleeve. Boring the sleeve into the cortical bone can require substantial time. It can also generate substantial heat, and cause the sharp sleeve cutting tip to become dulled. Not infrequently, perforation is abandoned due to substantial difficulty perforating the cortical bone. In some cases, the bone may become overheated. Further, the perforator and sleeve occasionally will fracture, become separated from the shank or drive coupling, and lodge in the bone.

A second disadvantage is that the device can cause discomfort for the patient when used for primary anesthesia when the cancellous bone is not pre-anesthetized by a block injection. The discomfort can be caused by heat from boring the hole with the large diameter sleeve. Discomfort can also be caused by the large combined perforator-sleeve volume intrusively perforating into the cancellous bone and displacing the fluids therein.

A third disadvantage is that controlled withdrawal of the perforator from the bony hole can be difficult. The user withdraws the perforator by forcefully pulling the connected handpiece in a direction away from the insertion site. However, during withdrawal the perforator is no longer rotating. When not rotating, the perforator tends to become tightly lodged in the bone, and can require moderate force to release. An additional moderate force is required to detach the perforator drive coupling from the sleeve. Further complicating the perforator removal, the sleeve frequently must be manually held in place to prevent it from pulling out of the bony hole with the perforator. The user's ability to forcefully extract the perforator in a controlled manner is impaired by the limited-access of the mandibular molar area. When adequate removal force is applied to the handpiece, the perforator typically releases suddenly. When the perforator releases suddenly, the handpiece tends to lurch away in an uncontrolled manner. The lurching handpiece and connected sharp perforator sometimes will strike other oral structures. Regardless of the outcome, forcefully removal of the perforator from the bone can be disconcerting to clinician and patient.

A fourth disadvantage is that fluid-flow through the sleeve can become obstructed by the sleeve tip pressing against cortical bone or other structures. Not uncommonly, the tip is inadvertently bored into contact with the lingual cortical bone. The jaw has a deceptively thin width, as the lingual cortical bone follows the buccal more closely than it would seem from palpating the lingual tissues. This occurs most frequently when placing the sleeve in a site distal to the second molar on the retromolar pad area.

A fifth disadvantage is that a substantial volume of fluid can leak back between the closely-fitted sleeve and the needle, and into the mouth. Because the needle is not tightly-fitted with the sleeve, there is substantial space for fluid backflow past the needle. When leakage occurs, the user loses the ability to track the volume of fluid actually injected into the cancellous bone. To stay within safe dosage limits, the user may elect to discontinue injecting the fluid before the desired effect is achieved. For example, if a user has dispensed a maximum safe volume of a local anesthetic, but the patient is not yet anesthetized, the user may elect to discontinue injecting further anesthetic. The reality may be that the patient simply has not received an effective volume of anesthetic into the cancellous bone due to leakage. As such, leakage can cause the injection to be ineffective. Further, patients find the taste of leaked anesthetics very offensive. Some users slide an endodontic stopper over the needle and against the hub to form a fluid-tight seal when the needle hub is pressed onto the sleeve orifice.

A sixth disadvantage is that the sleeve and drive-coupling can interfere with the workspace during dental procedures. The sleeve is typically located near the treated tooth, and typically protrudes about 5 millimeters out from the gingiva. As such, the sleeve presents a degree of interference, especially to the placement of rubber dam clamps, matrix bands, wedges, and so on.

A seventh disadvantage is that withdrawal of the sleeve can also be difficult from the bone. At most sites, the cortical bone tightly grips the sleeve, and substantial force is required to remove it. As with the perforator, forceful extraction of a sleeve from the bone can be disconcerting, especially if it releases suddenly, and in an uncontrolled manner.

An eighth disadvantage is that the sleeve can inadvertently become free in the mouth. Not infrequently, the cortical bone of some sites does not tightly grip the sleeve, such as sites distal to second molars. There is a risk that the sleeve might work itself free from the bony hole, and be swallowed or aspirated. If the user elects to remove the sleeve from such a non-secure site, then the sleeve must be reinserted into the bone later if an additional injection is required.

A ninth disadvantage is that the user must remember to remove the sleeve before the patient is dismissed. The sleeve is generally left in the site until the procedure nears completion, so substantial time can elapse between sleeve placement and removal. In addition, the sleeve is typically located distally to the operative site, and on the buccal side where it tends to become covered by cotton rolls and the patient's cheeks. It also may be covered by a rubber dam during the procedure. An out-of-sight sleeve must be remembered by the user at the sometimes-harried conclusion of a procedure. Sometimes the sleeve is forgotten, and the patient may be released from the office with the sleeve in mouth, representing a potential liability.

Aravena (US Appn 2006/0106363) shows a specialized intraosseous handpiece having a rotating hollow needle with a cutting tip for perforating cortical bone. The perforator needle is 24 gauge stainless steel having a medium OD of 0.5652 mm. The handpiece also has an injection syringe device for pressurizing fluid into the needle.

The cutting needle is rotated to perforate through the cortical bone. The syringe handpiece pressurizes fluid into the needle, and the needle injects fluid into the cortical bone. The needle may also be rotated to facilitate removal from bone.

The first disadvantage of the intraosseous handpiece is the substantial cost. The second disadvantage is that the medium diameter perforating needle can require excessive time and work to bore into hard cortical bone compared to a small diameter needle or perforator. Further, it does not have a small diameter perforator to lead with a pilot hole. As such, a significant amount of work is required to drill a 0.91 mm hole entirely through a thickness of cortical bone. The cutting tip of the needle also tends to become dull as drilling continues, exacerbating the difficulty.

A third disadvantage is that the medium diameter perforating needle is hollow, and does not have a supporting solid core. As such, the perforating needle has a risk of fracturing within the cortical bone. The risk of fracture is exacerbated by the substantial work required to bore the hole.

The above intraosseous injection devices and methods suffer from a number of disadvantages:
(a) Needle insertion into a small bony hole is difficult
(b) Perforating cortical bone to a medium to large diameter can require excessive time
(c) Perforating cortical bone to a large diameter can cause excessive heat
(d) Fluid can leak from between a closely fitted needle and sleeve
(e) A sleeve left protruding from a bony hole can interfere with the workspace
(f) A sleeve left in a bony hole can become loose in the mouth
(g) A sleeve left in a bony hole can be forgotten in the mouth
(h) Specialized intraosseous handpieces are costly

A device similar to the present bone bit would not have been appreciated prior to clinical experiences with current intraosseous injection devices and techniques. The present bit solves problems known primarily through the use of these current devices. It is unlikely that one would anticipate these problems prior to their use.

Further, demand for convenient and effective intraosseous injection devices and methods has been relatively low. Demand is just now beginning to increase due to recently developed, convenient, pre-anesthesia devices and methods. These devices reduce the time and discomfort associated with pre-anesthetising the gingiva for intraosseous anesthesia. Rapid and comfortable gingival pre-anesthesia methods are opening the door for intraosseous anesthesia to be used as a primary anesthesia method, rather than merely as an adjunctive method. As such, comfortable and rapid pre-anesthesia methods are beginning to increase demand for convenient, effective intraosseous devices.

### SUMMARY OF THE INVENTION

The system of the present invention is directed to an apparatus and method for injecting fluids through cortical bone and into cancellous bone without inserting an injector needle entirely through the cortical bone.

The cortical bone is considered to have a thickness extending from the gingival covering of the cortical bone to the border with the cancellous bone below. The thickness of the cortical bone may be considered to have a proximal portion that is proximal to the gingiva, called proximal bone, and a distal portion that is proximal to the cancellous bone, called distal bone.

The entire thickness of cortical bone is perforated to form a small-diameter bony conduit. The conduit may be considered to have a portion contained within the proximal bone called a proximal conduit, and a portion contained within the distal bone called a distal conduit. The term “proximal bone” is meant to include the proximal conduit contained therein.

An injector forms a fluid-tight seal with the gingiva about the conduit, with the proximal conduit, or with an enlarged access to the proximal conduit. Fluid from the injector is conducted directly through at least the distal conduit to the cancellous bone.

The apparatus includes a drill bit for boring the bony conduit through the entire thickness of cortical bone to the cancellous bone beneath. The bit has a small diameter perforator extending distally from a shank. The perforator has a cutting tip for perforating through the gingiva covering the cortical bone, through the cortical bone, and into the cancellous bone, thereby forming the bony conduit. The small diameter of the perforator facilitates forming the conduit with minimal work, such that the conduit may be bored quickly and with minimal heat.

In the preferred embodiment, a countersink is located on the bit several millimeters proximally from the distal end of the perforator, but several millimeters distally from the shank. The countersink has a larger diameter cutting tip than the perforator. As the perforator advances into the bone, the large-diameter countersink forms an access hole in the gingiva about the bony conduit called a gingival access. The gingival access has a large diameter relative to the conduit. The gingival access has a sufficiently large diameter to be readily locatable by the user for inserting a fluid injector.

If the countersink is advanced further, it will penetrate a distance into the proximal bone to form a bony access called an access seat. The bony access seat also has a sufficiently large diameter to be readily locatable by the user, and to readily receive the injector. However, the access seat has a sufficiently small diameter so as to be readily cutable by the countersink to a small depth into the proximal bone with minimal work.

The countersink is able to cut the access seat with minimal work because the cutting tip remains sharp when cutting only a small depth into proximal bone, the sidewall friction is minimal at a small depth, and the total volume of bone removed is minimal due to the small diameter of the access seat. As such, the perforator and countersink configuration minimize the work required to form both a bony conduit and a bony access seat.

The countersink configures the access seat for receiving the injector. The sizes and shapes of the access seat and the injector may be mated to form a sealing fit. The injector may be sized and shaped to form a fluid-tight seal by sealingly pressing against the proximal bone of the access seat. The term “proximal bone” also includes the access seat contained therein.

In the preferred method, a bony conduit is formed by advancing the rotating perforator to form a hole in the gingiva, and a conduit entirely through the thickness of cortical bone. Once the cortical bone is perforated, the perforator is advanced a distance into cancellous bone. As the perforator forms the bony conduit, the countersink contacts the gingiva that surrounds the perforator. As the bit is advanced further, a gingival access is formed by deepening the gingival hole until the countersink contacts the proximal bone.

The bit advances further until the countersink penetrates a distance into the proximal bone thickness, thereby forming an access seat within the proximal bone. The access seat is readily formed because its diameter and depth are sufficiently small for efficient boring. The bit is then withdrawn from the bone and gingiva.

To place the injector, the user readily locates the gingival access, and inserts the injector. User readily locates the bony access seat and inserts the injector. The diameters of the gingival access hole and of the bony access seat are sufficiently large to facilitate rapid and convenient injector insertion.

User presses the injector into the gingival access and the access seat in order to sealingly seat the injector therein. The size and shape of the access seat are closely matched to those of the injector so that the injector tip is sealingly fittable into the access seat. As such, a substantially fluid-tight seal may be formed. The injector is seated in the access seat in fluid communication with the bony conduit.

User activates the injector so that pressure is applied to the fluid, and the pressurized fluid is ejected from the injector. The seal between injector and access seat is substantially leak-tight and is able to contain the pressurized fluid from the injector, and thereby maintains the fluid pressure. Since the access seat is in fluid communication with the conduit, the pressurized fluid from the injector is directed through the access seat, through the bony conduit, and into the cancellous bone. After sufficient volume of fluid has been injected via the conduit into the cancellous bone, the injector is withdrawn from the site. If an additional injection is required, the process is readily repeatable by relocating the gingival access and access seat, sealingly reinserting the injector, and reinjecting the fluid.

Alternative methods involve forming a fluid-tight seal between the injector and any proximal surfaces surrounding the conduit, access seat, or gingival access, wherein the seal is formed in a manner that maintains a fluid communication with the conduit. The fluid-tight seal is formed by sealing pressure between the injector, or the injector hub, and the proximal surfaces. The proximal surfaces include the gingival surface, the walls of the gingival access, gingiva pushed into the access seat, the surface of the proximal bone, and the walls of the proximal conduit. For a given alternative method, the countersink may cut a gingival access only, but not an access seat, or the countersink may not be used at all, so that only the perforator is used to form the conduit.

Another aspect of the invention includes a gingival sleeve for enlarging the gingival access, or for beveling the bony access seat. Further, the sleeve may be left in the gingival access until the completion of a dental procedure to facilitate locating the gingiva access or the access seat. This is especially useful for sites where the gingiva is unusually thick over the access seat, or for sites in unattached gingiva.

As such, the term “access” may refer generally to injector-sealable surfaces of the either the gingiva, gingival access, bony access seat, or the proximal conduit.

In another aspect of the present invention, a method is provided for injecting fluid through a given thickness of a cortical bone having a cover of gingiva, wherein the thickness of cortical bone includes a proximal bone and a distal bone, comprising the steps of: boring a conduit through the gingiva and through the thickness of cortical bone, sealing an injector against the gingiva or proximal bone, wherein the injector is in fluid communication with the conduit, and injecting a fluid through at least the portion of the conduit that is in the distal bone.

### OBJECTS AND ADVANTAGES

Accordingly, several objects and advantages of my intraosseous injection system are to:
(a) minimize work to bore a bony conduit and injector access
(b) form a gingival access or bony access that facilitate convenient injector insertion
(c) provide an access that can form a fluid-tight seal with an injector
(d) minimize the risk of bit fracture
(e) minimize obstructions to injecting fluid into cancellous bone
(f) facilitate convenient reinjection
(g) minimize risk of neglecting to remove sharps from the mouth
(h) minimize patient anxiety with intraosseous injections

Further objects and advantages are to provide an economical bit for performing intraosseous injections. Still further objects and advantages will become apparent from a consideration of the ensuing description and drawings.

### DRAWING FIGURES

In the drawings, closely related figures have the same number, but different alphabetic suffixes.

FIG. 1 is a cutaway view showing a bit for forming a bony conduit and an access seat.

FIG. 2 is a partial cross-sectional view showing a bit forming a bony conduit and an access seat.

FIG. 3 is a partial cross-sectional view showing an injector sealingly seated in an access seat and injecting fluid through a bony conduit.

FIGS. 4A-4G are partial cross-sectional views showing various shapes of gingival access and bony access formed by various shapes of bits.

FIGS. 5A-5C are partial cross-sectional and cutaway views showing bits for boring an access seat having a beveled orifice.

FIGS. 6A-6D are cross-sectional and cutaway views showing a bit having a sleeve, the sleeve left in the tissue site after the bit is removed, and an injector inserted into the sleeve.

FIGS. 7A-7K are partial cross-sectional views showing various methods of obtaining a fluid-tight injector seal.

FIGS. 8A-8B are perspective and partial cross-sectional views of a detachable boring cylinder of the bit.

### REFERENCE NUMERALS IN DRAWINGS

| | | | |
|---|---|---|---|
| 10 | bit | 12 | perforator |
| 14 | countersink | 16 | shank |
| 18 | mark | 20 | nick |
| 22 | gingival access | 24 | gingiva |
| 26 | conduit | 28 | cortical bone |
| 30 | cancellous bone | 32 | proximal bone |
| 34 | distal bone | 36 | access seat |
| 38 | handpiece | 40 | injector |
| 42 | sleeve | 44 | bur |
| 46 | bevel | 48 | ring |
| 50 | large axle | 52 | small axle |
| 54 | shank offset | 56 | sleeve offset |
| 58 | axle offset | 60 | sleeve marking |
| 62 | small sleeve | 64 | hub |
| 66 | shank disk | 68 | countersink disc |
| 70 | blades | 72 | lumen |
| 74 | stopper | | |

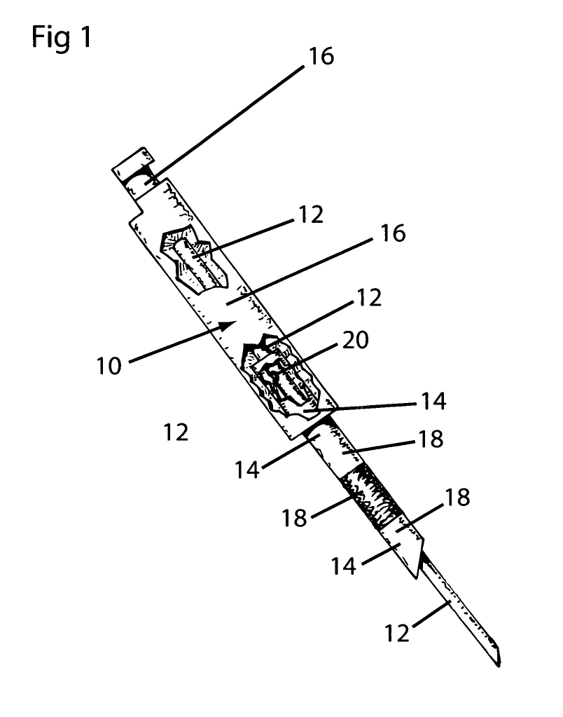

### Description—FIGS. 1 to 8

According to one aspect, the invention provides a method for injecting fluid through a given thickness of a cortical bone having a cover of gingiva, wherein the thickness includes proximal bone and a distal bone, comprising the steps of: boring a conduit through the gingiva and through the thickness of cortical bone, sealing an injector to the gingiva or to the proximal bone, wherein the injector is in fluid communication with the conduit, and injecting a fluid through at least the portion of the conduit that is in the distal bone.

According to another aspect of the invention, there is provided a drill bit 10 for use in the process of the invention, a typical embodiment of which is shown in FIG. 1. It is preferred that bit 10 is a rotary bit, such as for use in a standard dental latched contra-angle handpiece. However, bit 10 may be actuated by reciprocation, vibration, and so on.

The distal end of bit 10 comprises a small diameter perforator 12, wherein perforator 12 is for perforating through the gingiva, through the cortical bone, and into the cancellous bone. The diameter of perforator 12 is sufficiently small such that the cortical bone perforation is made quickly and with minimal heat. However, the diameter of perforator 12 is sufficiently large to resist fracture from the friction of perforating hard cortical bone.

It is preferred that perforator 12 comprises a solid-cylinder needle having a diameter within the range of 20 gauge to 32 gauge, and more specifically 26 gauge, or 0.4636 mm. However, perforator 12 may be hollow, and other diameters are also effective.

The distal end of perforator 12 comprises a bone cutter. It is preferred that the perforator 12 bone cutter comprises a sharply beveled surface similar to a needle bevel. However, the bone cutter may comprise electroplated diamonds, flutes with cutting edges, ceramics, and so on.

It is preferred that perforator 12 is comprised of nickel-titanium alloy to maximize resistance to fracture, and for durable cutting-edge sharpness. However, perforator 12 may be comprised of hardened stainless steel, or other materials.

It is preferred that the cylindrical sides of perforator 12 are smooth to minimize friction. However, the sides may be textured, fluted, and so on, such as to facilitate debris removal.

A countersink 14 is located concentrically along the axis of bit 10 several millimeters proximally from the distal end of perforator 12. Countersink 14 is for forming a gingival access, and for forming an access seat in the proximal bone, wherein the access seat is configured for sealingly receiving an injector. Countersink 14 has a somewhat greater diameter than perforator 12, and is for forming a gingival access and an access seat in the proximal bone.

The gingival access and the access seat that are cut by countersink 14 must each have a sufficiently large diameter to be conveniently locatable by the user. However, the access seat also has a sufficiently small diameter to be readily cutable without excess friction, and in a short period of time. As such, the diameter of countersink 14 must balance these objectives, so it is not too large, and not too small.

The preferred diameter of countersink 14 is 20 gauge, having OD 0.9081 mm. A 0.9081 mm diameter countersink 14 is readily able to cut an access seat to the preferred depth of 3 mm in a short time period. The cutting tip tends to remain very sharp when cutting only to such a shallow depth as 3 mm, even when cutting in to hard proximal bone. Further, with an access seat depth of 3 mm, the sidewall friction is minimal, and the total volume of bone removed is minimal. As such, the configurations of perforator 12 and countersink 14 minimize the work required to form both a bony conduit and a bony access seat.

It is further preferred that countersink 14 comprises a 20 gauge hollow needle having ID 0.603 mm. As such, countersink 14 is able to concentrically contain a proximal portion of perforator 12 within the lumen, while a distal portion of perforator 12 extends several millimeters from countersink 14. As such, a conduit cut by concentric perforator 12 and an access seat cut by countersink 14 are also concentric with one another. It is preferred that countersink 14 is comprised of nickel-titanium alloy to maximize resistance to fracture, and for durable cutting-edge sharpness. However, countersink 14 may be 19 gauge, 20 gauge, and so on.

The distal end of countersink 14 comprises a bone cutter for cutting an access seat in the proximal bone. It is preferred that the countersink 14 bone cutter comprises a sharp needle bevel. However, the bone cutter may comprise electroplated diamonds, flutes with cutting edges, ceramics, and so on.

Countersink 14 connects to a shank 16. Shank 16 is configured externally as a standard bur shank for connecting to a latched dental contra-angle handpiece. It is preferred that countersink 14 extends 9 mm from the distal end of shank 16. It is preferred that perforator 12 extends 5 mm distally beyond countersink 14. It further preferred that shank 16 is 15 mm in length, such that 14 mm of shank 16 is fully inserted into a standard handpiece head, and 1 mm of shank 16 extends distally from the head. The diameter of shank 16 is identical to that of standard latch burs, or 2.35 mm. As such, the total length of bit 10 is 29 mm. However, other lengths are effective.

The surface of perforator 12 develops substantial friction when boring the conduit against the cortical bone, and has a proportional risk of fracture. However, the length of perforator 12 is shorter relative to cortical perforators of other intraosseous systems. As such, the shorter length of perforator 12 minimizes the risk of fracture relative to other perforators. Further, because countersink 14 cuts an access seat a partial distance into the cortical bone thickness, the remaining cortical bone thickness requiring perforation is reduced by the depth of the access seat. For perforator 12 to perforate through the remaining partial cortical bone thickness, the length of perforator 12 extending distally from countersink 14 may be less than the total cortical bone thickness. In effect, countersink 14 carries, protects, and reinforces perforator 12 during a portion of the penetration of perforator 12 into the cortical bone. This reduces the risk of perforator 12 fracture during perforation.

The length of countersink 14 is shown having visually distinguishable marks 18 to function as depth guides during the boring of the access seat. Marks 18 may comprise differentiating and distinguishing markings, such as surface textures, paint, stain, anodizing, indentations, ridges, and so on. Marks 18 may also include the portions of countersink 14 which occur between distinguishable markings, but have the appearance of the original countersink 14 surface. A first mark 18 at the distalmost 3 mm of countersink 14 has an original appearance. In a distal-to-proximal direction, the second mark 18 comprises a darkly pigmented band 3 mm in length. The third mark 18 comprises a 3 mm band having an original appearance, and ending at shank 16.

Marks 18 indirectly indicate the penetration of countersink 14 into the proximal bone. In use, when user notices resistance to advancement of bur 10 into the bone when countersink 14 initially contacts proximal bone, user may note which individual mark 18 is showing immediately above the gingiva. As countersink 14 bores into the proximal bone, a portion of a mark 18 will disappear into the gingiva. As such, the user is able monitor the depth of penetration of countersink 14 into the proximal bone as it bores an access seat.

The cutaway view of shank 16 shows a portion of countersink 14 retentively encased by shank 16. Perforator 12 extends a greater distance proximally into shank 16 than countersink 14 such that the shank 16 material contacts and retentively encases a substantial surface area of perforator 12, as well as of countersink 14. Such retentive encasing secures the transfer of rotational force from shank 16 directly to perforator 12 and directly to countersink 14. It is preferred that the retentive surface area portions of perforator 12 and countersink 14 have surface texturing to enhance retention with shank 16.

It is preferred that perforator 12 is secured only by direct connection to shank 16, and is not secured by connection to countersink 14. However, perforator 12 may be secured by connection to countersink 14.

It is preferred that shank 16 is comprised of a plastic having a degree of deformability, such as nylon. As such, the stress of forceful engagement between the drive mechanism of a dental handpiece and shank 16 will tend to cause deforming wear on shank 16. Such shank 16 wear will discourage multiple uses of bit 10, and encourage single-use and disposal. Further, it is preferred that bit 10 is not autoclavable, and that shank 16, or other parts thereof, will melt at autoclave temperatures. However, bit 10 may be autoclavable for multiple uses.

The proximal portion of perforator 12 that is encased in shank 16 and enclosed within countersink 14 is shown having a nick 20. Nick 20 is an area where the shaft of perforator 12 is intentionally weakened for safety. As such, when forces acting upon perforator 12 are sufficient to fracture the shaft, then the perforator 12 shaft is likely to fracture at nick 20 rather than at other points along the shaft. As such, the likelihood of a shaft fracture distal to nick 20 is decreased. If a fracture occurs at nick 20, then recovery of the remaining distal shaft from the site is facilitated. However, nick 20 is effective in other locations along the length of the shaft of perforator 12.

Measuring from the distal end of shank 16, countersink 14 is embedded 4 mm within shank 16, and perforator 12 is embedded 7 mm into shank 16 beyond countersink 14. Nick 20 is 3 mm from the distal end of shank 16, and 1 mm from the proximal end of countersink 14. As such, nick 20 is 12 mm from the distal end of countersink 14, and 17 mm from the distal end of perforator 12. It is preferred that perforator 12 is not connected to countersink 14 or shank 16 at any point distal to nick 20. However, other embedding lengths are effective.

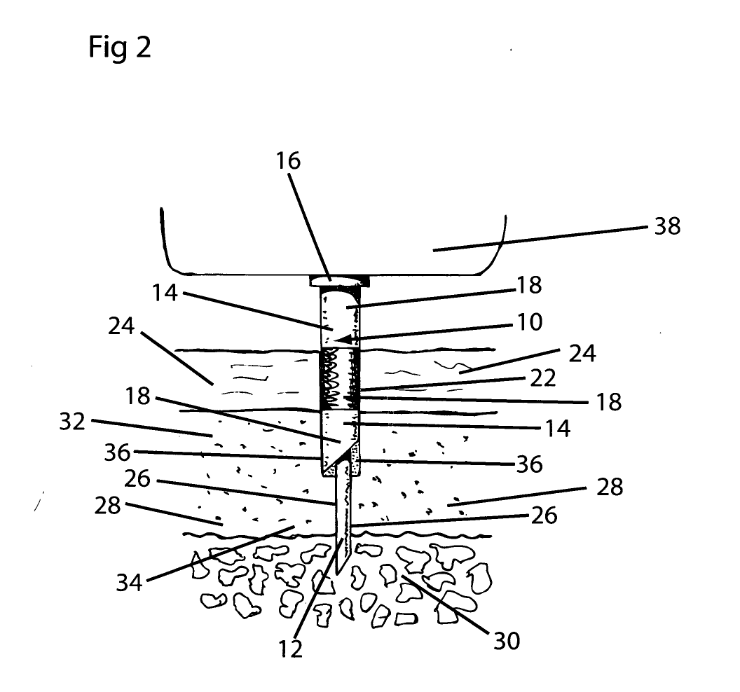

FIG. 2 shows perforator 12 after having formed a gingival access 22 in a gingiva 24 and a conduit 26 through a cortical bone 28 and into a cancellous bone 30. Perforator 12 readily penetrates cortical bone 28 because the diameter is small, and the cutting end is sharp.

For this discussion, the total thickness of cortical bone 28 is considered to be comprised of a proximal partial thickness and a distal partial thickness. The proximal partial thickness is toward gingiva 24, called proximal bone 32, and the distal partial thickness is toward cancellous bone 30, called distal bone 34. The thickness of proximal bone 32 is considered to be the same or greater than the thickness of distal bone 34. The term “proximal bone 32” is considered inclusive of the portion of conduit 26 that passes through proximal bone 32.

Countersink 14 is shown having enlarged the diameter of gingival access 22, and cutting an access seat 36 into proximal bone 32. Countersink 14 is readily able to cut access seat 36 into proximal bone 32 because the diameter is sufficiently small, despite being large relative to the diameter of perforator 12. Further, only a short depth of penetration into proximal bone 32 is required to form a sealable access seat 36. The preferred depth of penetration range for access seat 36 is 1-4 mm, with a preferred depth of 3 mm. As such, only a small volume of proximal bone 32 is removed to form access seat 36. Proximal bone 32 is considered to inclusive of access seat 36.

When cortical bone 28 is perforated through to cancellous bone 30, patients occasionally experience discomfort. A wider-diameter perforation has a greater likelihood of causing discomfort due to the subsequent displacement of a larger volume of fluid within cancellous bone 30. Such patient discomfort is minimized with bit 10 because the wider-diameter countersink 14 does not perforate through cortical bone 28 and into cancellous bone 30 when cutting access seat 36.

FIG. 2 shows an access seat 36 having a cylindrical portion with parallel opposing sidewalls. Such a cylindrical portion facilitates secure positioning of an injector, and a fluid-tight seal between access seat 36 and an injector. Access seat 36 is also shown having an end portion that is cut perpendicularly with respect to the cylindrical walls. The end-portion of access seat 36 can function to enhance the fluid-tight sealing between the injector and access seat 36, and can act as a depth-stop for the injector. A fluid-tight seal between an injector and access seat 36 ensures that a given volume of fluid ejected from the injector will substantially be directed into conduit 26 and conducted into cancellous bone 30. A fluid-tight seal also minimizes undesirable fluid leakage into the mouth.

Marks 18 provide a reference to assist the user in determining the depth of countersink 14 penetration into proximal bone 32 while cutting access seat 36. When countersink 14 has bored through gingiva 24 and makes initial contact with proximal bone 32, the user is able to observe which mark 18 is showing immediately above the surface of gingiva 24. As access seat 36 is cut into proximal bone 32, user can estimate the cutting depth by observing the number of marks 18 that disappear below the surface of gingiva 24 during the cut. For example, if each mark 18 has a length of 3 mm, and one mark 18 has disappeared into gingiva 24, then access seat 36 has been cut to a depth of 3 millimeters into proximal bone 32.

Shank 16 is shown inserted into a handpiece 38, and engaged with the drive mechanism thereof. It is preferred that handpiece 38 comprises a latch contra-angle slowspeed dental handpiece. However, handpiece 38 may comprise a friction grip handpiece, or any other type of handpiece. It is preferred that bit 10 is rotated at less than 20,000 rpm during use.

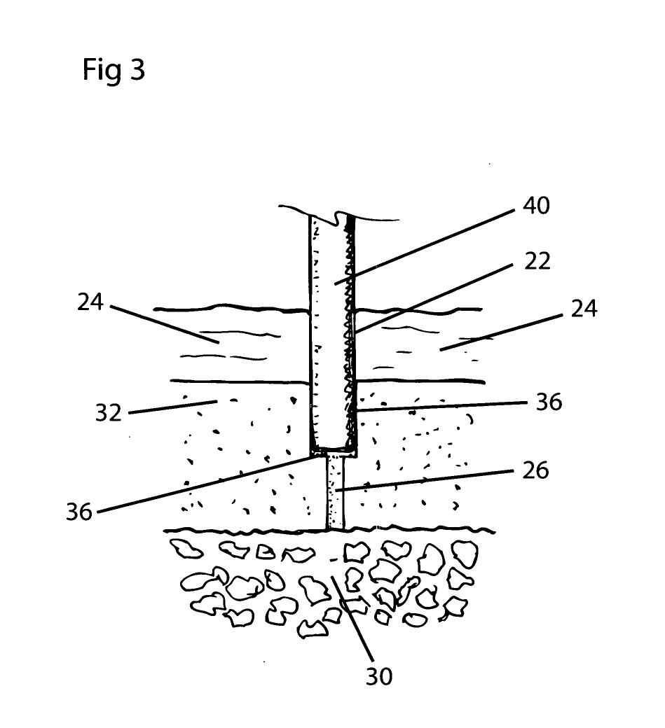

FIG. 3 shows an injector 40 inserted through gingival access 22 of gingiva 24 and sealingly engaged into access seat 36 of proximal bone 32. Access seat 36 is configured to mate with injector 40 in such a way that a fluid-tight seal is formed between access seat 36 and injector 40.

It is preferred that injector 40 forms a fluid-tight seal with access seat 36 by sealingly pressing against the parallel cylindrical walls of access seat 36. However, injector 40 may sealingly press against the bottom of access seat 36, against portions of access seats 36 having configurations different from that shown in FIG. 3, against other portions of proximal bone 32, or against gingiva 24. Further, injector 40 may sealingly press against gingiva 24 that is displaced into access seat 36, such that gingiva 24 is sealingly interposed between injector 40 and the surfaces of access seat 36.

Injector 40 shown in FIG. 3 comprises a cylinder having smooth, parallel sides, and a blunt tip. The cylindrical parallel sides of injector 40 exert even pressure against the cylindrical parallel sides of access seat 36, thereby facilitating a fluid-tight seal. However, the tips of various injectors 40 may be tapered, polished and rounded, bulbous, threaded, corrugated, beveled, flared, and so on. Further, injectors 40 may have tip configurations that facilitate sealingly engaging gingiva 24, or pulling gingiva 24 into access seat 36 to enhance fluid-tight sealing between injector 40 and access seat 36.

When injector 40 is sealingly seated into access seat 36, fluid expressed from injector 40 is contained by the fluid-tight seal between injector 40 and access seat 36, and is thereby directed into conduit 26, and into cancellous bone 30.

It is preferred that a 0.9081 mm diameter access seat 36 is mated with a 22 gauge injector 40 with OD 0.7176 mm. However, a 0.9081 mm access seat 36 may be mated with a 20 gauge injector 40 with 0.9081 OD mm, or with a 21 gauge injector 40 with OD 0.8192 mm, and so on. Further, a 0.8192 mm access seat 36 may be mated with a 21 gauge injector 40 with OD 0.8192 mm, or a 22 gauge injector 40 with OD 0.7176 mm, or a 23 gauge injector 40 with OD 0.6414 mm, and so on.

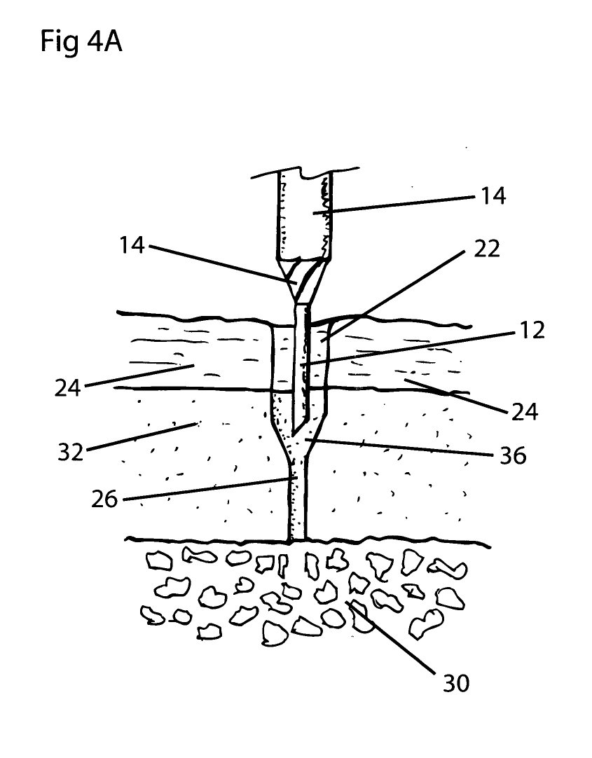

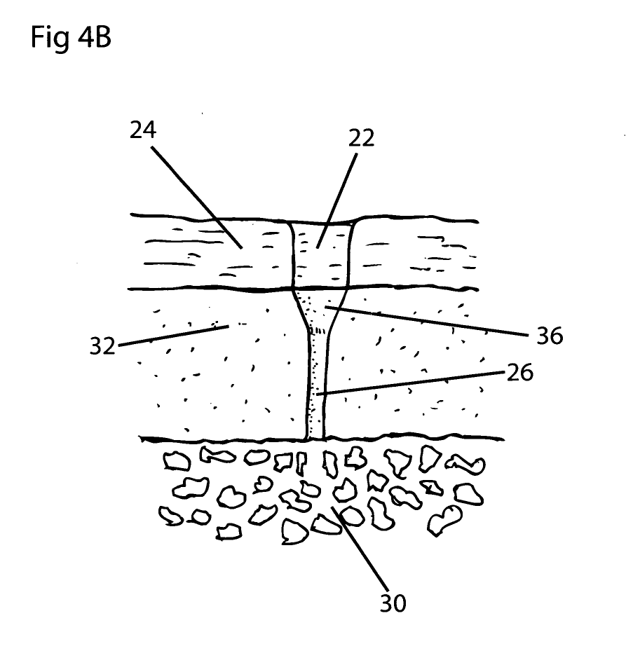

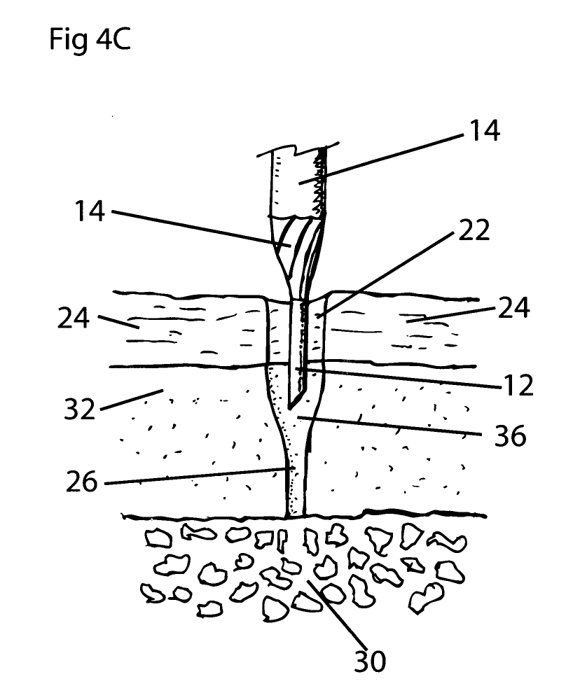

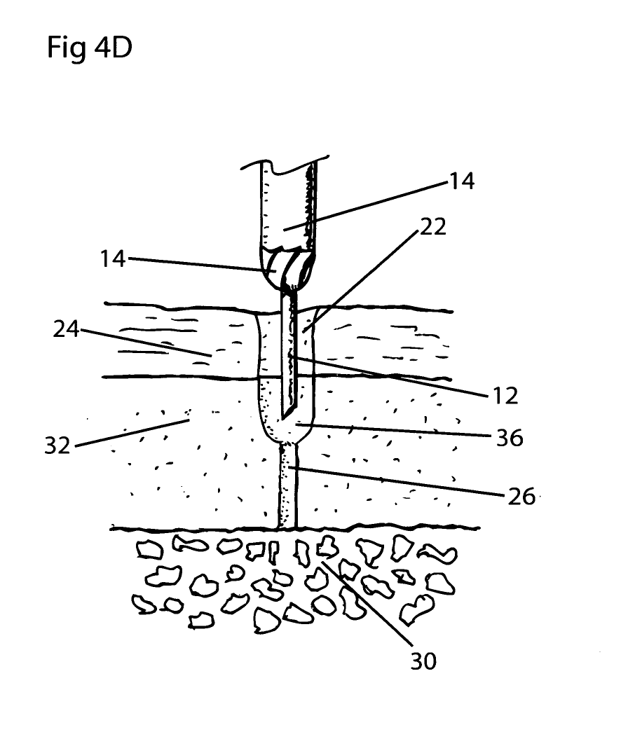

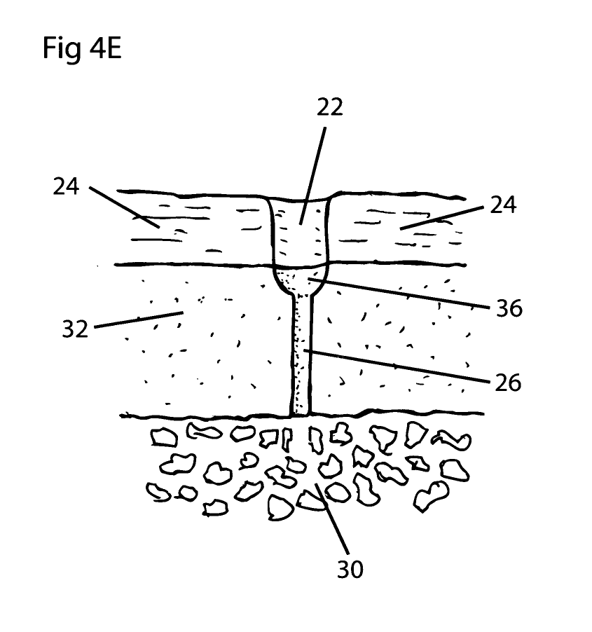

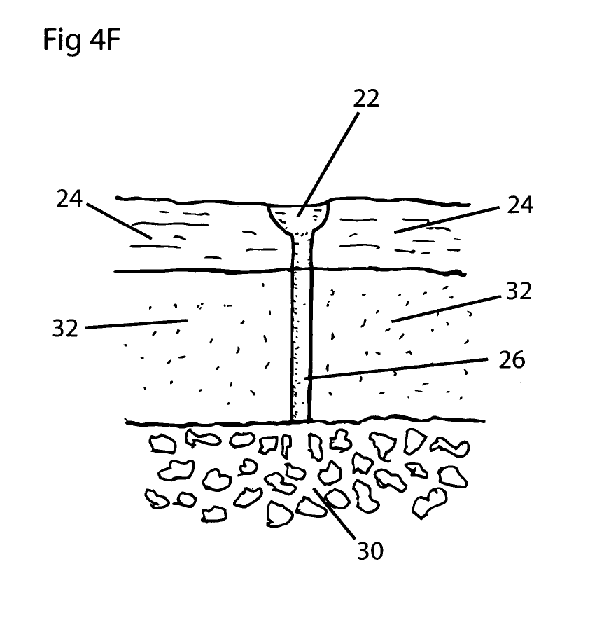

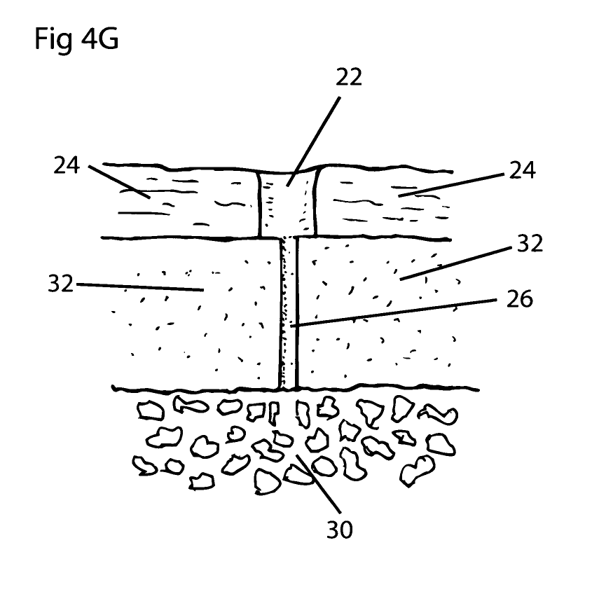

FIGS. 4A-4H show examples of various shapes of countersinks 14 and the access seats 36 bored thereby.

FIG. 4A shows an access seat 36 having a tapered end-stop and parallel cylindrical walls in proximal bone 32. Gingival access 22 also has cylindrical walls. A countersink 14 having a tapered cutter is shown. Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 4B shows an access seat 36 having a tapered end-stop in proximal bone 32, but cylindrical walls are formed only in gingival access 22. When injector 40 is sealingly seated with access seat 36 or gingiva 24, fluid expressed from injector 40 is contained by the fluid-tight seal, and is thereby directed into conduit 26, and into cancellous bone 30.

FIG. 4C shows an access seat 36 having a funnel-shaped end-stop in proximal bone 32. Cylindrical walls are formed in gingival access 22. A countersink 14 having a funnel-shaped cutter is also shown. Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 4D shows an access seat 36 having a curved end-stop and cylindrical walls in proximal bone 32. Gingival access 22 also has cylindrical walls. Use of a round-tipped injector 40 can facilitate forming a fluid-tight seal. A countersink 14 having a rounded cutter is shown. Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 4E shows an access seat 36 having a curved end-stop in proximal bone 32. Gingival access 22 has cylindrical walls. When injector 40 is sealingly seated with access seat 36 or gingiva 24, fluid expressed from injector 40 is contained by the fluid-tight seal, and is thereby directed into conduit 26, and into cancellous bone 30.

FIG. 4F shows a gingival access 22 having a curved end-stop. The curved end-stop is cut a partial-depth into gingiva 24, and does not penetrate to proximal bone 32. Injector 40 can form a liquid-tight seal by compressing the remaining gingiva 24 against proximal bone 32. When injector 40 is sealingly seated with gingiva 24, fluid expressed from injector 40 is contained by the fluid-tight seal, and is thereby directed into conduit 26, and into cancellous bone 30.

FIG. 4G shows a gingival access 22 cut to the surface of proximal bone 32 by a countersink 14. Access seat 36 is not formed. When injector 40 is sealingly seated with access seat 36 or gingiva 24, fluid expressed from injector 40 is contained by the fluid-tight seal, and is thereby directed into conduit 26, and into cancellous bone 30.

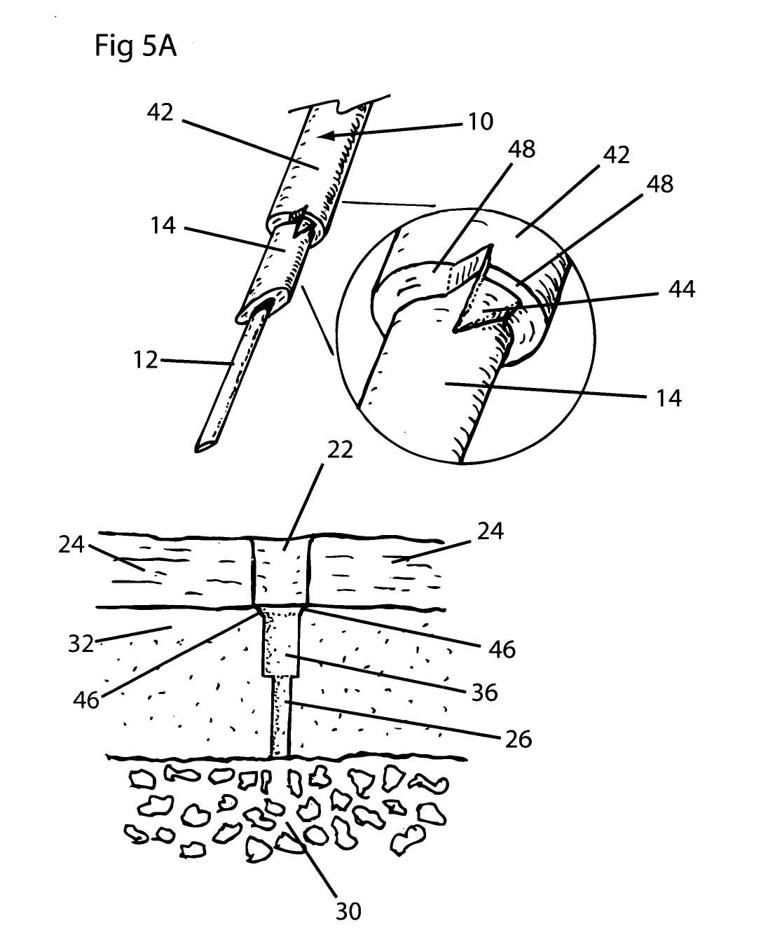

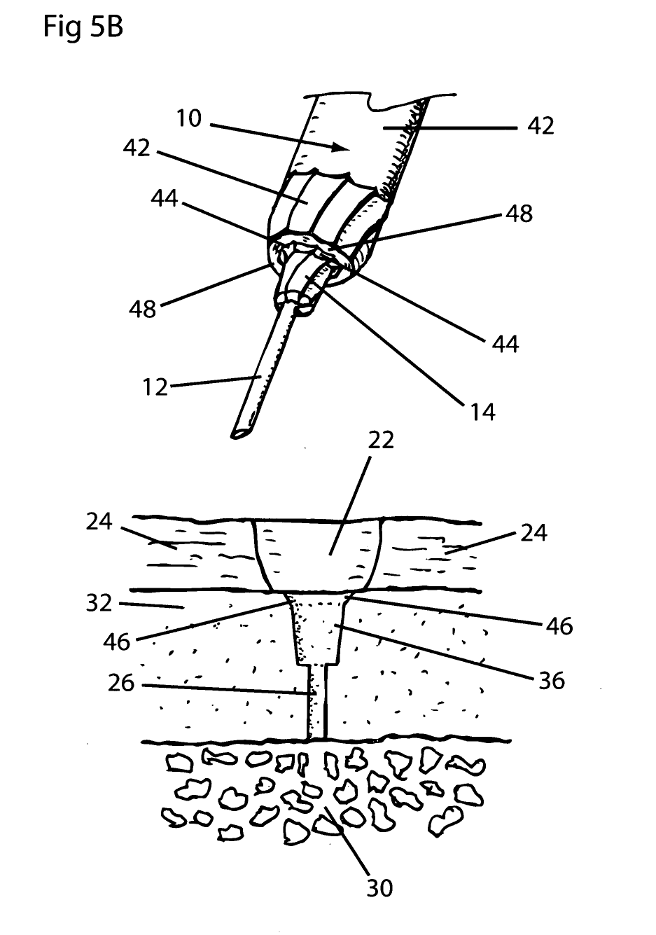

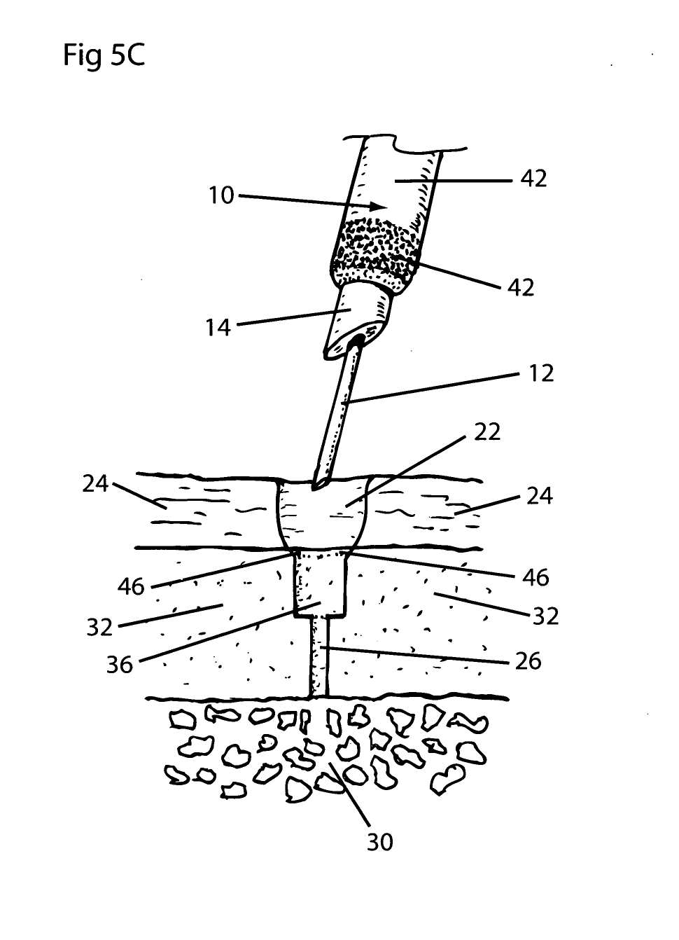

FIGS. 5A-5C show bits 10 for forming an access seat 36 having a beveled orifice.

FIG. 5A shows a bit 10 having a sleeve 42 covering the proximal portion of countersink 14. Sleeve 42 covers countersink 14 proximally to the distal end of shank 16, but does not cover the distal end of countersink 14. The non-covered portion of countersink 14 extends a distance distally from sleeve 42, such as 3 mm. The distal extension portion of countersink 14 not covered by sleeve 42 includes the cutting tip that forms access seat 36. For a 9 mm length countersink 14 where the distalmost 3 mm of countersink 14 remains uncovered by sleeve 42, sleeve 42 has a length of 6 mm.

A bur 44 extends distally from the distal end of sleeve 42. Bur 44 has sufficient hardness to cut proximal bone 32 such that it is able to bevel the bony orifice of access seat 36 to form a bevel 46 thereon. A single bur 44 may extend from the distal end of sleeve 42, or a multiplicity of burs 44 may be spaced about the circumference of sleeve 42.

Bevel 46 provides a wider target for the initial contact of injector 40 during insertion, and thereby facilitates injector 40 insertion into access seat 36. The slope of bevel 46 guides injector 40 to become centered as it inserts into access seat 36. Bevel 46 therefore reduces injector 40 insertion interferences. However, injector 40 may form a fluid-tight seal by pressing directly against the surfaces of bevel 46, without further insertion into access seat 36.

Bur 44 on the distal end of sleeve 42 is shown having a leading edge that is substantially perpendicular to the length of sleeve 42, and comprises the cutting edge. During clockwise rotation of bit 10, the perpendicular leading edge of bur 44 makes initial contact with proximal bone 32 prior to contact by the remaining portions of bur 44. However, bur 44 may be configured as a cutting flute, an abrasive-coated surface, and so on.

A notch is shown adjacent to the perpendicular leading edge of bur 44, wherein the notch indents proximally into sleeve 42. Such a bur 44 notch may be convenient for manufacturing, may facilitate the removal of debris from the cutting edge, and may facilitate cutting gingiva 24 to widen gingiva access 22. However, a bur 44 may be present on sleeve 42 without such a notch.

The distalmost portion of bur 44 tapers to a point. The greatest width of the bur 44 is proximal to, and is connected to, the distal end of sleeve 42. The greatest width of bur 44 is less than the total cylinder-wall width of the distal end of sleeve 42. The bur 44 connection to sleeve 42, which is where bur 44 has its greatest width, therefore comprises only a partial width of the total width of the distal end of sleeve 42.

Other than bur 44 and the bur 44 notch, the remaining surface of the distal end of sleeve 42 is substantially perpendicular to the sleeve 42 cylinder wall. The total cylinder-wall width of the distal end of sleeve 42 may be considered to have an inner portion that is adjacent to countersink 14, called an inner ring, and an outer portion, called ring 48. Bur 44 may be considered to be connected to the inner ring, such that the width of bur 44 at the connection defines the width of the inner ring. The remaining outer width thickness of the distal end of sleeve 42 comprises ring 48. Ring 48 has a larger diameter than the inner ring of sleeve 42. Ring 48 therefore comprises the portion of the distal end having the largest diameter.

The bur 44 notch is shown eliminating a small portion of the inner ring, and a small portion of ring 48 adjacent to bur 44. As such, the surface of ring 48 is substantially uninterrupted. However, bur 44 notch may eliminate a portion of the inner ring only, leaving ring 48 uninterrupted, or the notch may not be present at all.

When bur 44 cuts a bevel 46 into proximal bone 32, ring 48 simultaneously displaces or cuts gingiva 24, and comes to rest against the uncut surface of proximal bone 32 outside of bevel 46. Ring 48 will not cut proximal bone 32. As such, ring 48 serves as a depth-stop to limit the insertion of countersink 14 into proximal bone 32.

It is preferred that a sleeve 42 having a bur 44 is affixed to countersink 14, or to shank 16. As such, sleeve 42 is removed from the injection site when the remainder of bit 10 is withdrawn. Once sleeve 42 is withdrawn from an access seat 36 having a bevel 46, then bevel 46 is able to facilitate insertion of injector 40.

However, sleeve 42 may be removable from bit 10 such that sleeve 42 may remain in gingival access 22. When a sleeve 42 with burs 44 are left in gingival access 22, the burs 44 nest within bevel 46 to stabilize the position of sleeve 42 in gingival access 22.

Selecting a removable sleeve 42 of a given length can effectively control the depth of access seat 36. Selecting a shorter sleeve 42 will yield a deeper access seat 36 because it leaves a greater length of countersink 14 exposed. Selecting a longer sleeve 42 will yield a shallow-depth access seat 36.

Further, sleeve 42 can enlarge access seat 36 by boring into proximal bone 32 with a bur 44. Sleeve 42 may remain inserted into enlarged access seat 36, such that a proximal portion of sleeve 42 protrudes from proximal bone 32 for maintenance of gingival access 22 patency. A bur 44 for enlarging access seat 36 has a cutting surface having a width equal to the thickness of the sleeve 42 cylinder wall. As such, a sleeve 42 for enlarging access seat 36 has no ring 48 depth-stop.

Bevel 46 may be cut by other means, such as by a beveled shank offset 54 connected to a small axle 52. Further, bevel 46 may be cut by a second countersink 14 located proximally on bit 10 from a first countersink 14, and having a greater diameter that the first countersink 14, and so on.

Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 5B shows a bit 10 having an affixed sleeve 42. Sleeve 42 has a multiplicity of burs 44 for forming a bevel 46. The distal end surface of sleeve 42 has a continuous, uninterrupted ring 48 for limiting the depth of access seat 36.

The sleeve 42 cylinder wall also has an outer wall portion of greater diameter than the ring 48 cylinder-wall portion. The distal end of the outer wall portion of sleeve 42 is beveled, and has a multiplicity of cutting-flutes. The beveled outer-wall cutter is for cutting a bevel into gingival access 22, such that gingiva 24 is cut back from the orifice of access seat 36. As such, gingiva 24 is less likely to slump into access seat 36, or interfere with the insertion of injector 40.

Bit 10 has a countersink 14 having cutting flutes on the lateral sidewalls. The countersink 14 is slightly tapered, wherein the diameter of the proximal end is slightly greater than the diameter of the distal end. The flattened, distal end of countersink 14 also has cutting flutes for boring access seat 36. As such, tapered countersink 14 bores an access seat 36 having slightly tapered walls.

It is preferred that a tapered access seat 36 has a 0.04 taper. For a 0.04 tapered access seat 36 having an orifice diameter of 0.9081 mm, the diameter at a depth of 3 mm is 0.7995 mm. As such, a 21 gauge injector 40 having an OD 0.8192 mm would be readily insertable into the 0.9081 mm orifice of access seat 36. When such an injector 40 is inserted to near the end-stop of tapered access seat 36, injector 40 engages and sealingly presses against the sidewalls of tapered access seat 36. The gradual taper of tapered access seat 36 facilitates a smooth, ever-tightening, insertion of injector 40 such that a fluid-tight seal is formed.

Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 5C shows a bit 10 with an affixed sleeve 42 having a rounded distal edge. The distal surface of sleeve 42 is coated with abrasive particles. The sleeve 42 abrasive surface is for enlarging gingival access 22 and for forming a bevel 46. Perforator 12 and countersink 14 have rapid-cutting beveled tips. For a 0.9081 mm OD countersink 14, it is preferred that sleeve 420D is 1.473 mm.

The abrasive distalmost 0.5 mm of sleeve 42 is coated with fine abrasive grit. Fine grit abrasives, such as 30 μm diamonds, cut bone more slowly that medium or coarse grit. The distal end of sleeve 42 therefore cuts bone more slowly than the beveled cutting tips of perforator 12 and countersink 14.

As such, bit 10 bores readily into proximal bone 32 prior to abrasive sleeve 42 contacting proximal bone 32. When abrasive sleeve 42 contacts the thin edge of the access seat 36 orifice, a small volume of proximal bone 32 is readily removed to form bevel 46. However, as bevel 46 widens, substantial cutting resistance is quickly encountered. For example, a 0.2 mm width bevel 46 provides substantial cutting resistance to abrasive sleeve 42. As such, bevel 46 provides substantial resistance to further insertion of bit 10. The bit 10 insertion resistance provides tactile feedback for the user, signaling that bevel 46 has been formed and countersink 14 has bored a sufficient depth into proximal bone 32.

The distalmost 0.5 mm end of sleeve 42 is coated with fine abrasive grit for cutting bevel 46. A 2 mm long band of 100 μm medium grit abrasive is located immediately proximal to the fine grit abrasive of the distal end. The medium grit abrasive is for enlarging gingival access 22, wherein the medium grit cuts gingival access 22 faster than fine grit. The medium grit abrasive efficiently cuts gingiva 24 back from the orifice of access seat 36. As such, gingiva 24 is less likely to slump into access seat 36, or interfere with the insertion of injector 40.

Alternatively, the bevel 46 cutting efficiency may be limited by only sparsely coating the distalmost 0.5 mm of sleeve 42 with medium grit abrasive. Further, bevel 46 cutting efficiency may be limited by the placement of fine cutting flutes over the distalmost 0.5 mm.

When injector 40 is sealingly seated with access seat 36 or gingiva 24, fluid expressed from injector 40 is contained by the fluid-tight seal, and is thereby directed into conduit 26, and into cancellous bone 30.

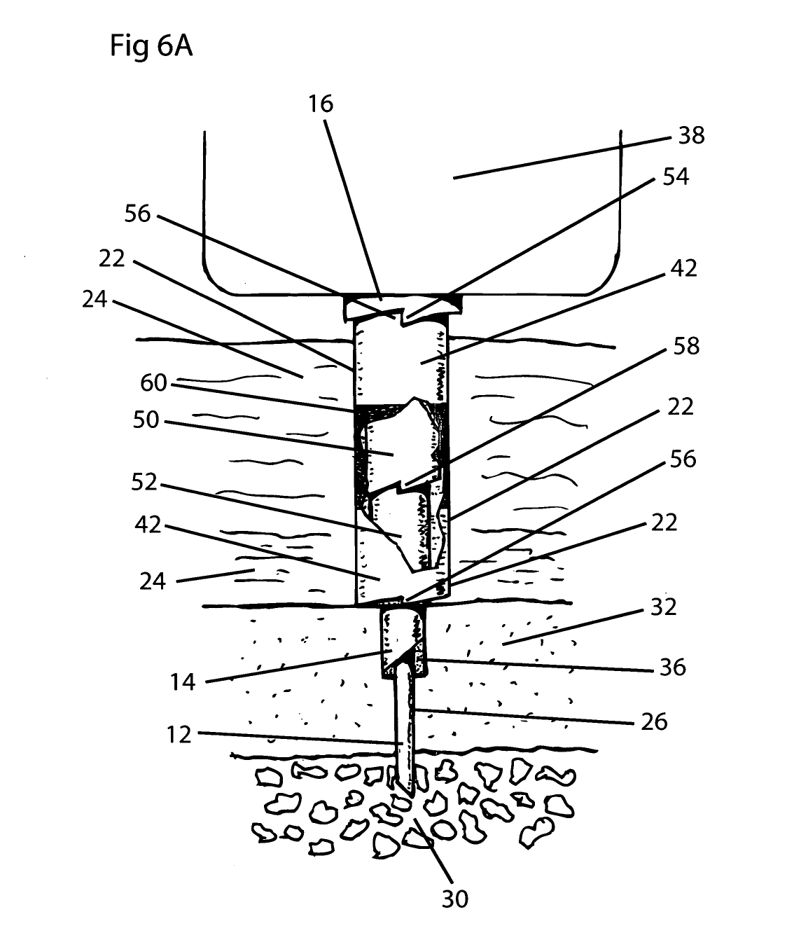

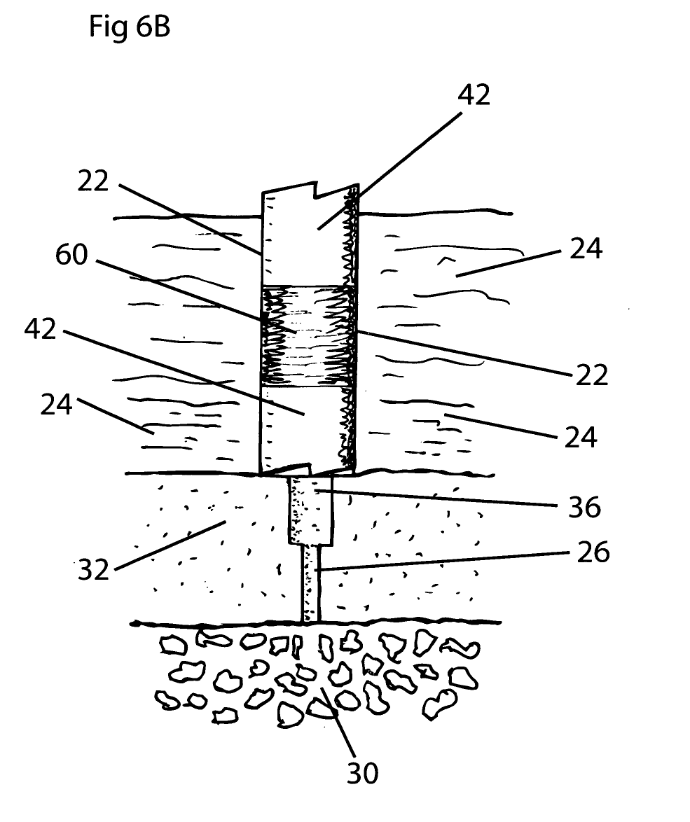

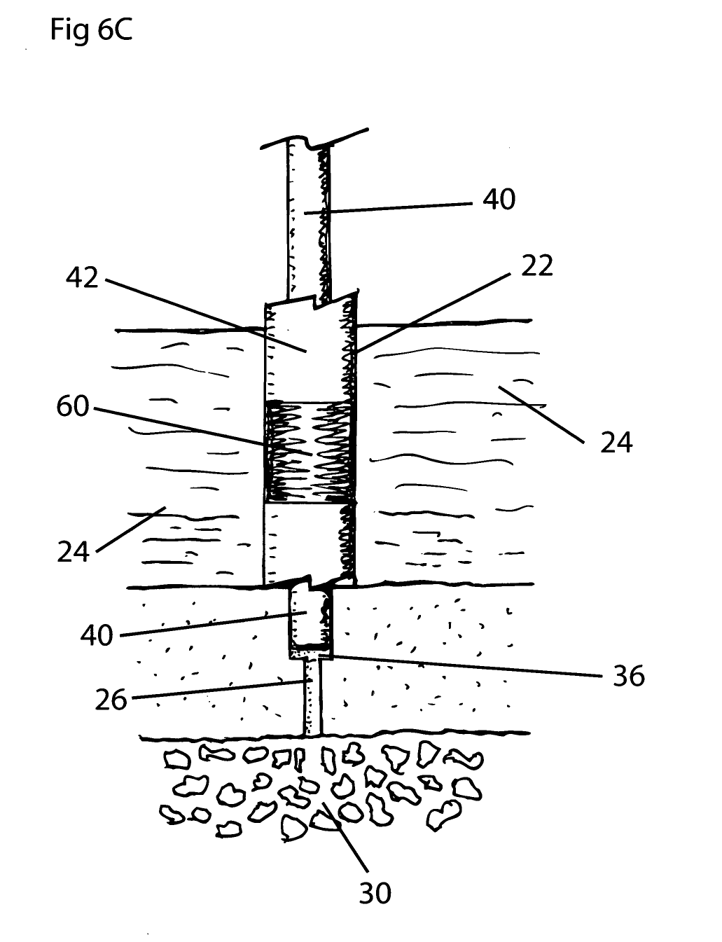

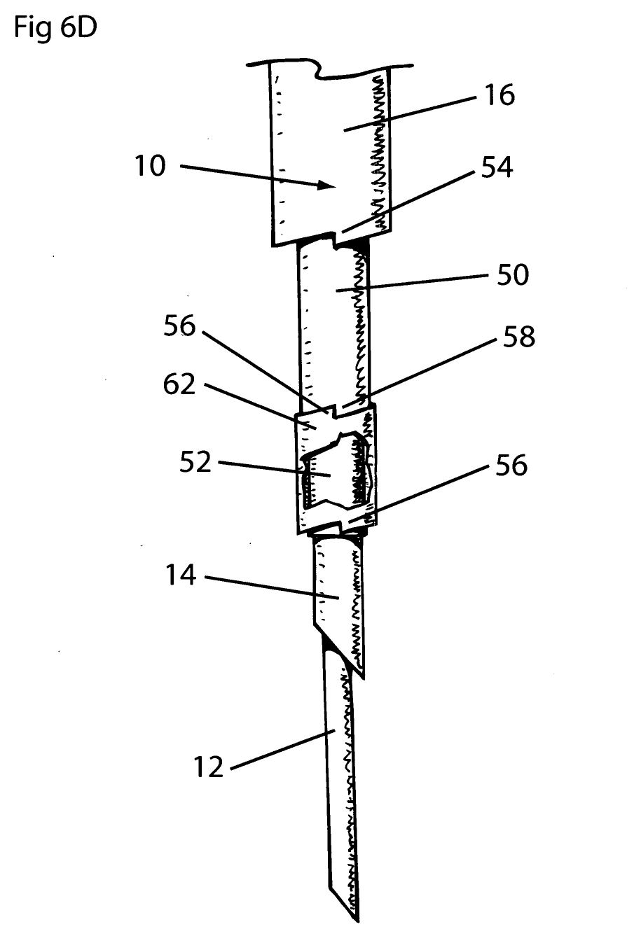

FIGS. 6A-6D show sleeve 42 used for enlarging gingival access 22. Sleeve 42 is also used for maintaining gingival access 22. Sleeve 42 comprises a cylinder that covers a portion of the length of countersink 14.

FIG. 6A shows a sleeve 42 that is removably mounted on a bit 10 as countersink 14 bores an access seat 36 into proximal bone 32. Shank 16 is inserted into a handpiece 38. The sleeve 42 shown covers countersink 14 proximally to the distal end of shank 16, but does not cover the distal end of countersink 14. The non-covered portion of countersink 14 extends a distance distally from sleeve 42, such as 3 mm. The distal extension portion of countersink 14 not covered by sleeve 42 includes the cutting tip that forms access seat 36. For a 9 mm length countersink 14 where the distalmost 3 mm of countersink 14 remains uncovered by sleeve 42, then sleeve 42 has a length of 6 mm.

Gingival access 22 is shown after having been enlarged by sleeve 42. Gingival access 22 is enlarged but not beveled. The distal end of sleeve 42 is in contact with the surface of proximal bone 32. It is preferred that the distal end of the removable sleeve 42 has sufficient hardness to cut and enlarge gingival access 22, but not proximal bone 32. As such, as countersink 14 bored access seat 36 to a given depth, such as 3 mm, sleeve 42 cuts through gingiva 24 and comes into contact with the surface of proximal bone 32, or the periosteum thereover. Sleeve 42 does not cut proximal bone 32, and stops countersink 14 from advancing further into proximal bone 32. Sleeve 42 therefore functions as a depth-stop for countersink 14. However, sleeve 42 may be non-cutting and may enlarge gingival access 22 by simply displacing gingiva 24 as sleeve 42 rotates toward proximal bone 32. Further, sleeve 42 may or may not remove the periosteum over proximal bone 32. Sleeve markings 60 facilitate measuring the depth of gingiva 24.

Sleeve 42 is shown removably fitting over a large axle 50, shown in the cutaway. Large axle 50 coaxially covers a proximal portion of countersink 14, and has a larger diameter than countersink 14. The ID of removable sleeve 42 is fitted to be slightly larger than the diameter of large axle 50, such that sleeve 42 is readily removable from large axle 50. Sleeve 42 is removably retained on large axle 50 by friction between the closely fitted surfaces. Sleeve 42 retention on large axle 50 may be enhanced by glycerin, mineral oil, and so on, between sleeve 42 and large axle 50.

A small axle 52, shown in the cutaway, covers a portion of countersink 14 that is distal to large axle 50. The preferred length of large axle 50 is 3.5 mm, and the preferred length of small axle 52 is 2.5 mm. The total combined lengths of large axle 50 and small axle 52 is 6 mm. Small axle 52 does not cover a distal portion of countersink 14, such as the distal 3 mm of countersink 14. Further, sleeve 42 removably covers both large axle 50 and small axle 52 from the distal of shank 16 to within 3 mm of the distal end of countersink 14. As such, the distal 3 mm of countersink 14 can cut access seat 36 to a depth of 3 mm into proximal bone 32 before the distal end of small axle 52 contacts proximal bone 32, thereby stopping further penetration of countersink 14.

The diameter of small axle 52 is smaller than the diameter of large axle 50, but larger than the diameter of countersink 14. Sleeve 42 does not directly contact small axle 52 because the diameter of small axle 52 is smaller than the ID of sleeve 42.

It is preferred that the diameter of large axle 50 is 1.270 mm, or 18 ga, and the diameter of small axle 52 is 1.067 mm, or 19 ga. A sleeve 42 that removably fits over a 1.270 mm large axle 50 is 15 gauge, having an OD of 1.829 mm and an ID of 1.372 mm. Such a sleeve 42 has adequate clearance for unimpeded insertion and removal of a 0.8192 mm OD 21 gauge injector 40, a 0.7176 mm OD 22 gauge injector 40, and so on.

However, a 1.270 mm diameter large axle 50 may extend from shank 16 to within 3 mm of the distal end of countersink 14, so that no small axle 52 is present. A 1.270 mm large axle 50 may be removably covered by a 15 gauge, 1.829 mm OD and 1.372 mm ID sleeve 42. Further, sleeve 42 may removably cover countersink 14 directly, where no large axle 50 or small axle 52 are present. For a 0.9081 mm diameter countersink 14, a sleeve 42 that directly covers countersink 14 is 17 gauge, OD 1.473 and ID 1.067 mm, or larger. Yet further, sleeve 42 may be non-removably affixed to countersink 14, to large axle 50, to shank 16, and so on.

Sleeve 42 is rotated as countersink 14 and large axle 50 are rotated. It is preferred that sleeve 42 rotation is driven by a shank offset 54 at the distal end of shank 16. Shank offset 54 is a “tooth” on shank 16 for engaging and rotating sleeve 42 as shank 16 is rotated. Sleeve 42 has a mated sleeve offset 56 to engage shank offset 54 during rotation.

It is further preferred that two mated sets of shank offsets 54 and sleeve offsets 50 are spaced 180° opposite one another on the bit 10 axis. However, a single mated set or multiple mated sets may be present, or sleeve 42 may be rotated by a very close frictional fit against large axle 50 or countersink 14, and so on. Sleeve 42 is shown having a sleeve offset 56 at both the proximal and distal ends. A distal sleeve offset 56 can facilitate cutting gingiva 24 or proximal bone 32.

An axle offset 58 is located at the distal end of large axle 50. Axle offset 58 is for driving the rotation of a small sleeve that may be mounted over small axle 52.

It is preferred that the outer surface of sleeve 42 has retentive features to enhance retention to gingival access 22. As such, the positioning of sleeve 42 is stabilized within gingival access 22, thereby decreasing the likelihood of inadvertent dislodgment of sleeve 42.

The preferred sleeve 42 retentive feature comprises an adhesive surface for adhering to gingival access 22. It is further preferred that the sleeve 42 adhesive surface comprises a gel capable of absorbing moisture from gingival access 22, and thereby becoming sticky and adhesive to gingival access 22. Other effective sleeve 42 retentive features include surface texturing, lapped ridges, directional scales or quills that slip over gingival access 22 during rotational insertion of sleeve 42 but engage gingival access 22 once rotation has ceased, and so on.

It is preferred that sleeve 42 is comprised of a bioabsorbable material, such as gelatin, vegetable gel, gelatin coated cellulose, hydroxypropylmethylcellulose, carbohydrate, paper, karaya gum, glycerin, dipropylene glycol, propylene glycol, lecithin, any combination thereof, and so on. As such, sleeve 42 would dissolve away within a short time period if left in a gingival access 22, or if swallowed or aspirated. However, sleeve 42 may be comprised of elastomers, plastic, metal, ceramic, composites, and so on.

It is preferred that sleeve 42 has a color which contrasts with the color of gingival 24. A contrastingly colored sleeve 42 facilitates location of sleeve 42 and gingival access 22, and helps the user notice sleeve 42 at the end of the procedure for sleeve 42 removal.

The outer surface of sleeve 42 is shown having a sleeve marking 60 for indicating the depth of penetration of sleeve 42. Sleeve marking 60 comprises a band of contrasting appearance over a given length of sleeve 42. Sleeve marking 60 is shown as a pigmented band covering the distal 3 mm, and the proximal 3 mm of a 6 mm sleeve 42 is a non-pigmented band.

Sleeve 42 may be left in gingival access 22 after the remainder of bit 10 is removed from the site. Sleeve 42 maintains the patency of gingival access 22 and visually contrasts with the surrounding gingiva 24. Sleeve 42 thereby facilitates locating gingival access 22 and access seat 36 and the insertion of injector 40 into gingival access 22 and access seat 36.

In FIG. 6A, countersink 14 has advanced to cut gingival access 22 and access seat 36 in proximal bone 32. Sleeve offset 56 on the proximal end of sleeve 42 was engaged by shank offset 54 on the distal end of shank 16. Sleeve 42 has cut gingiva 24 to widen gingival access 22, and advanced until the distal end contacted proximal bone 32. The distal end of sleeve 42 did not cut proximal bone 32. Sleeve 42 has thereby stopped the penetration of countersink 14 into proximal bone 32. As such, sleeve 42 limits the depth of access seat 36 into proximal bone 32, and functions as a countersink 14 depth-stop. Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 6B shows sleeve 42 inserted into gingival access 22 after the remainder of bit 10 is withdrawn. When sleeve 42 remains inserted into gingival access 22, sleeve 42 provides a straight, non-interfering insertion conduit through gingival access 22 to proximal bone 32, or to access seat 36.

In some areas of the mouth, the gingiva is loosely attached to the underlying proximal bone 32, and does not remain affixed in a predictable position relative to the proximal bone 32. As such, when a gingival access 22 is formed in loosely attached gingiva 24, the gingival surface portion of gingival access 22 may be out of alignment with access seat 36. Such misalignment of gingival access 22 with access seat 36 can obstruct injector 40 during insertion through gingival access 22 and into access seat 36. This problem is exacerbated with a large thickness of loose gingiva 24.

Sleeve 42 prevents gingiva 24 from interfering by slumping into the injector 40 path of insertion. Further, the diameter of sleeve 42 is sufficiently large to provide insertion clearance for injector 40. As such, sleeve 42 can provide a predictable and convenient insertion pathway for injector 40 through gingival access 22.

For example, when a 6 mm length sleeve 42 is fully inserted into a gingival access 22 having a depth of 5 mm, then the proximal end of sleeve 42 will be 1 mm above the surface of gingiva 24. As such, sleeve 42 and will substantially maintain the patency of the gingival access 22. Further, the contrasting color of sleeve 42 facilitates the location of gingival access 22 by a user. Sleeve 42 therefore facilitates location of gingival access 22 and insertion of injector 40. The contrasting color also serves to remind the user to remove sleeve 42 from gingival access 22 at the end of the procedure.

Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

Sleeve 42 has sufficient length to protrude from gingival access 22 in most locations, further facilitating the location of gingival access 22 for injector 40 insertion. With sleeve 42 positioned in gingival access 22, injector 40 may be conveniently and rapidly inserted into access seat 36 as needed, as shown in FIG. 6C. Since injector 400D is typically smaller than the ID of sleeve 42, injector 40 readily moves in and out of sleeve 42 without dislodgment of sleeve 42.

However, the ID of sleeve 42 may be the same or smaller that the OD of injector 40, such that sleeve 42 sealingly fits over injector 40 to form a fluid-tight seal for containing an injected fluid within access seat 36 or conduit 26.

When sleeve 42 is no longer needed, sleeve 42 is readily removable by grasping with cotton pliers or a hemostat to withdraw from gingival access 22. If sleeve 42 was forgotten by the user and left in gingival access 22, or inadvertently swallowed or aspirated by a patient, sleeve 42 harmlessly disintegrates within in a few hours.

It is anticipated that bit 10 and sleeve 42 would have good patient acceptance because the use is comfortable for the patient, conduit 26 is very small, and no sharp parts remain in the mouth except during direct use.

Sleeve markings 60 facilitate measuring the depth of gingiva 24. Perforator 12 has bored conduit 26. When injector 40 is sealingly seated with access seat 36 or gingiva 24, fluid expressed from injector 40 is contained by the fluid-tight seal, and is thereby directed into conduit 26, and into cancellous bone 30.

FIG. 6D shows a small sleeve 62 removably mounted over small axle 52, shown in the cutaway. Small sleeve 62 may be substituted for sleeve 42 in cases where the depth of gingiva 24 is only 2 to 3 mm. It is preferred that small sleeve 62 is 2.5 mm in length, and has a color that contrasts with gingiva 24. The distal end of sleeve 62 extends distally to the distal end of small axle 52, leaving the distalmost 3 mm of countersink 14 uncovered.

When a 2.5 mm length small sleeve 62 is fully inserted into a gingival access 22 having a depth of 3 mm, then the proximal end of small sleeve 62 is 0.5 mm below the surface of gingiva 24. As such, small sleeve 62 and will substantially maintain the patency of the gingival access 22. The contrasting color of small sleeve 62 facilitates the location of gingival access 22 by a user.

Small sleeve 62 therefore facilitates location of gingival access 22 and insertion of injector 40. The contrasting color also serves to remind the user to remove small sleeve 62 from gingival access 22 at the end of the procedure.

Small sleeve 62 has a sleeve offset 56 at the proximal end that is engaged with axle offset 58 at the distal end of large axle 50. Small sleeve 62 has a sleeve offset 56 at the distal end that cuts gingiva 24 for enlarging gingival access 22. It is preferred that small sleeve 62 is 16 gauge, having an OD of 1.651 mm and an ID of 1.194 mm. As such, the ID of small sleeve 62 fits closely with the diameter of a 1.067 mm, 19 gauge small axle 52. However, other gauges and lengths are effective. Shank 16 is shown with an unused shank offset 54. Perforator 12 is at the distal end of bit 10.

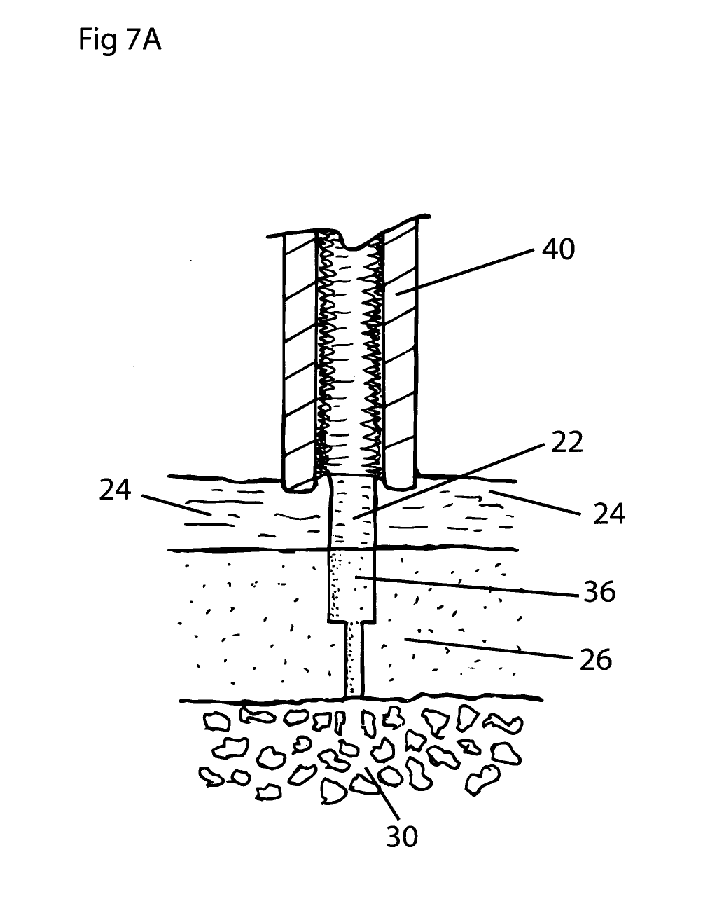

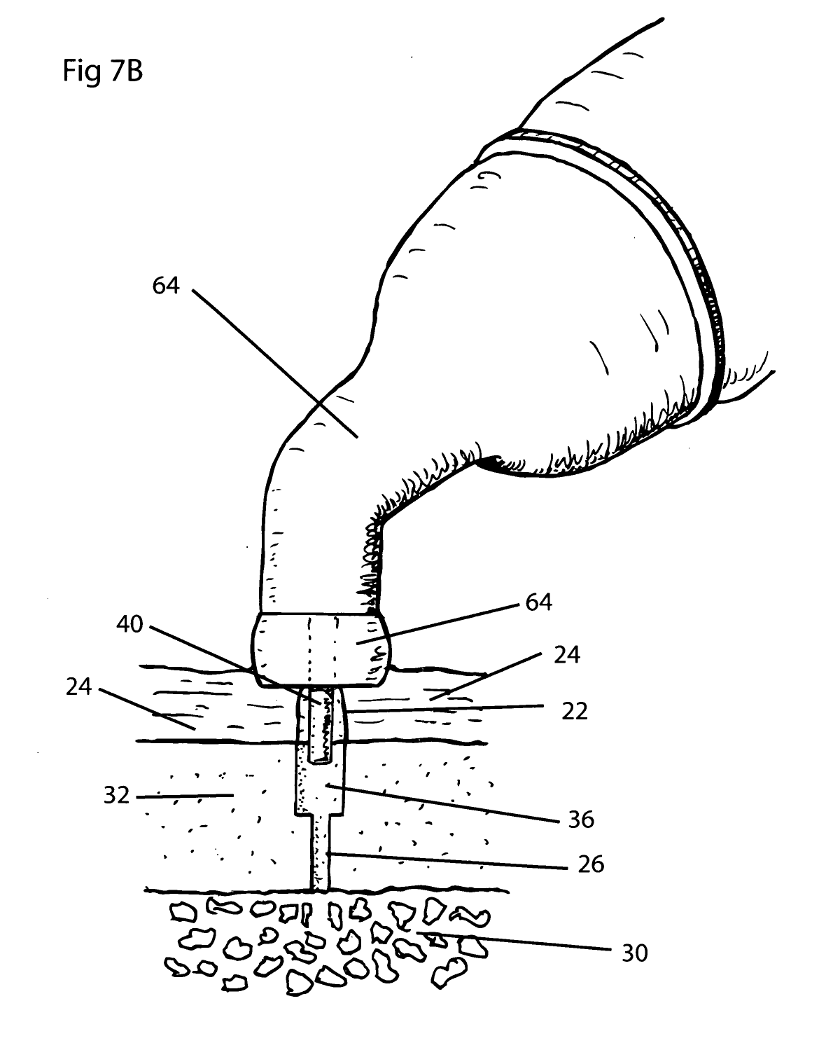

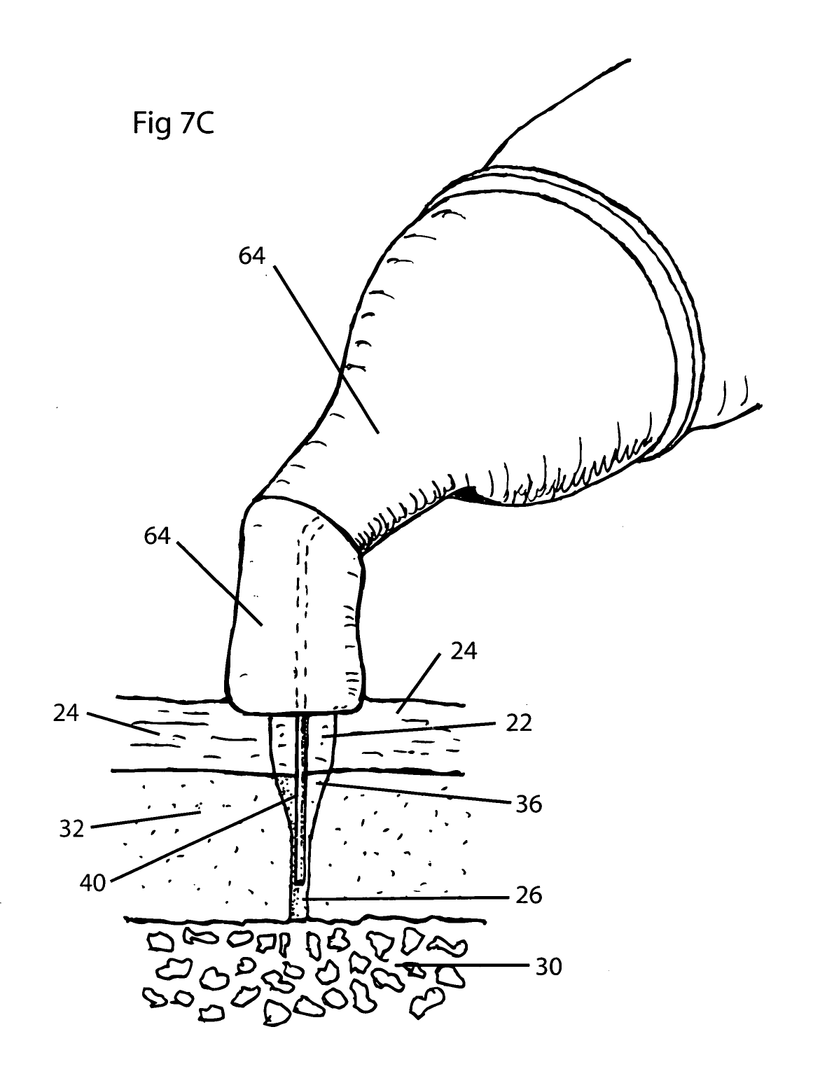

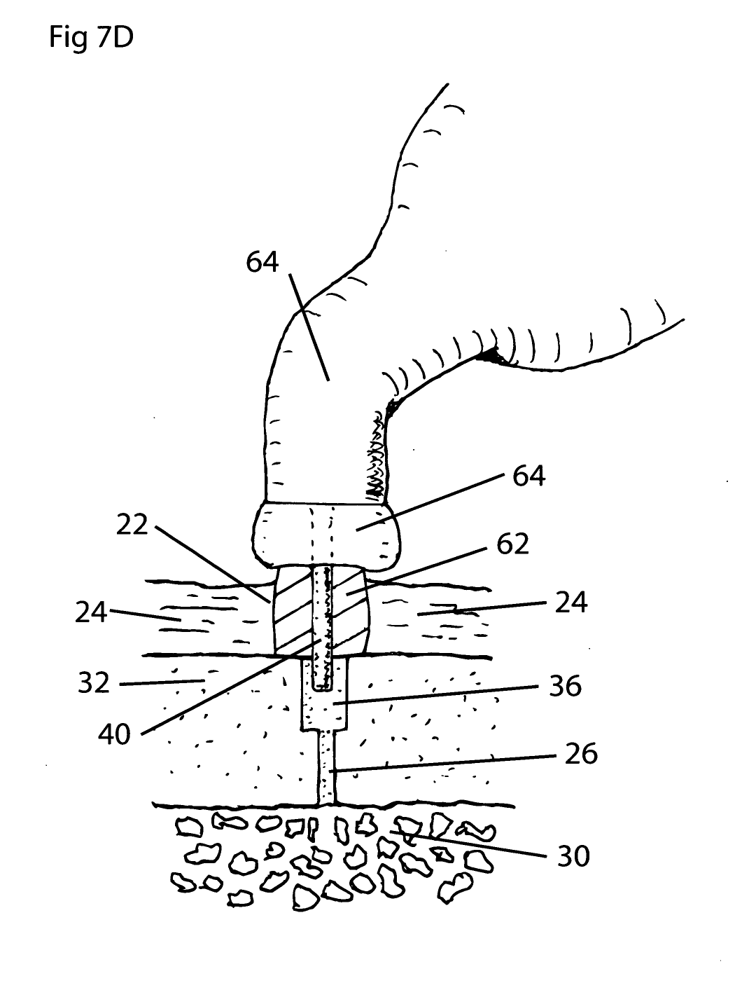

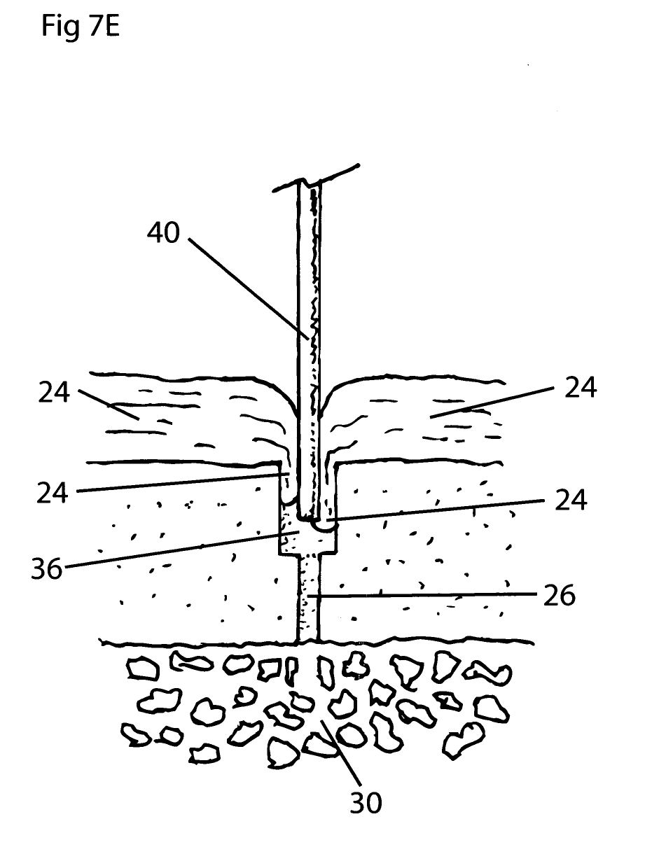

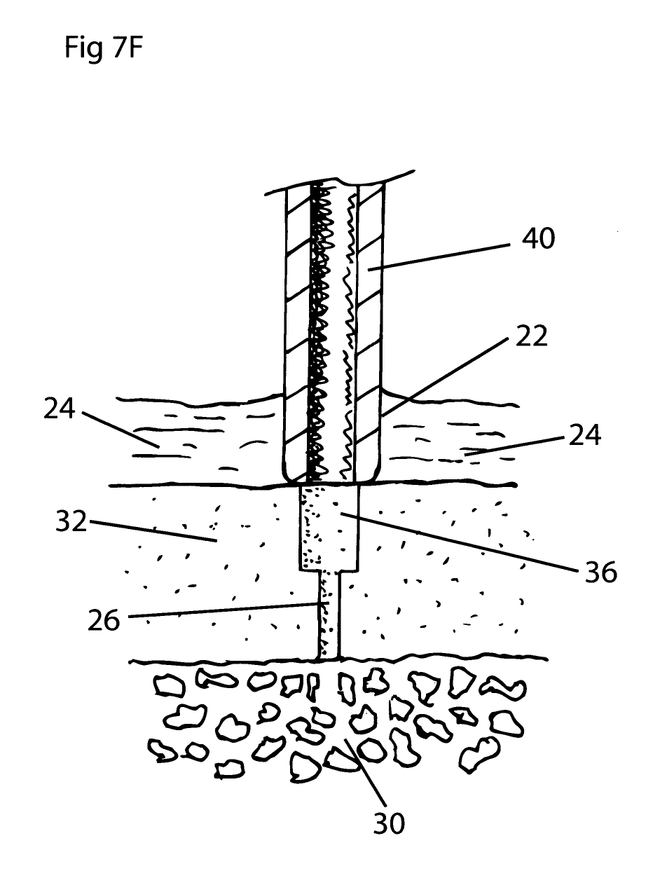

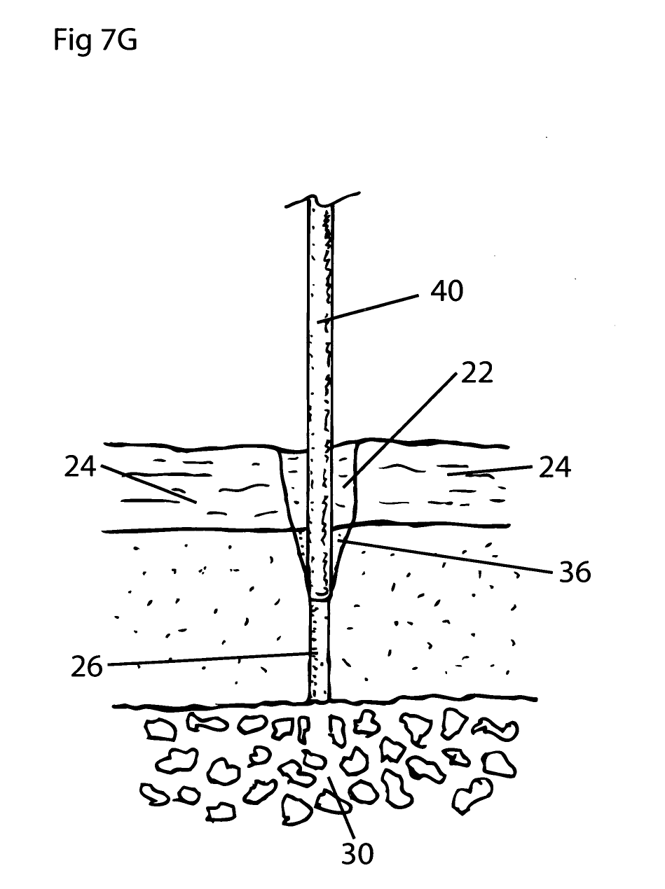

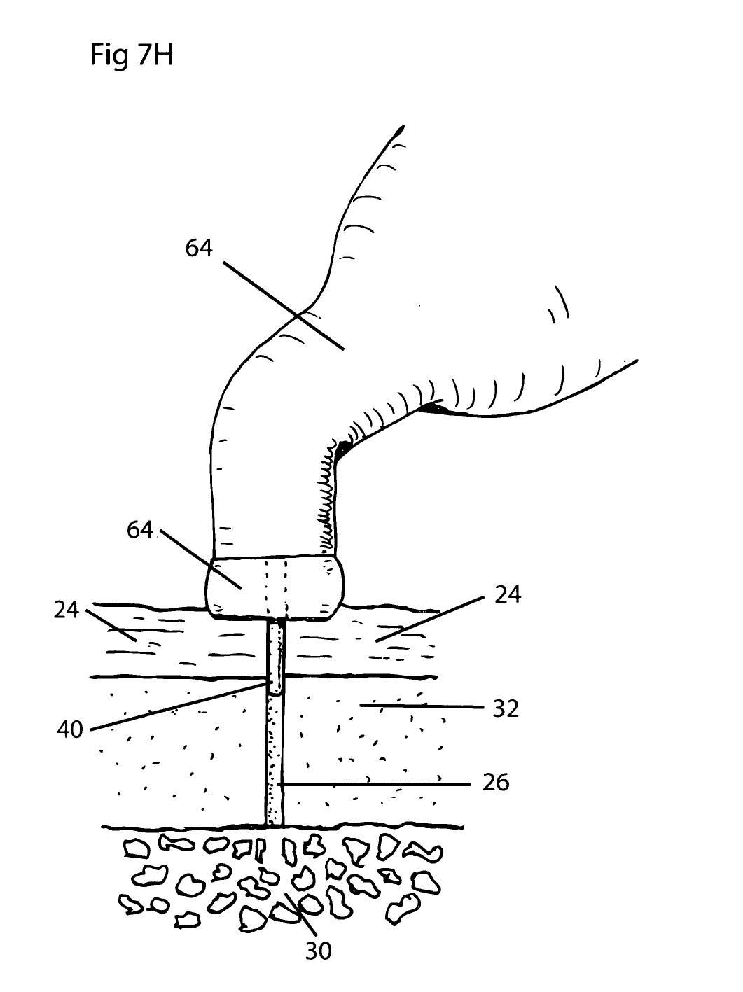

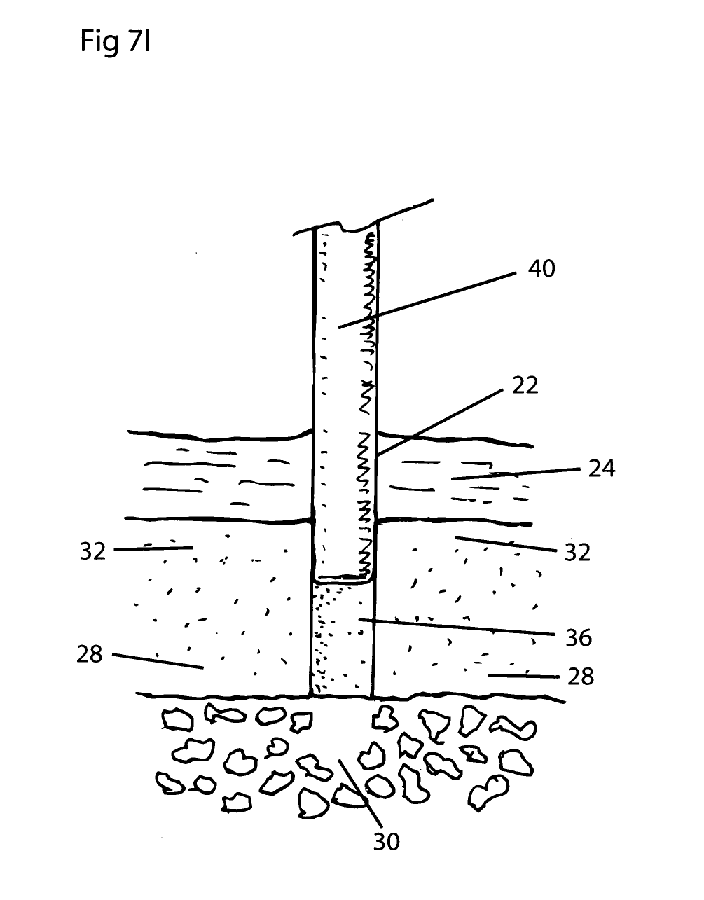

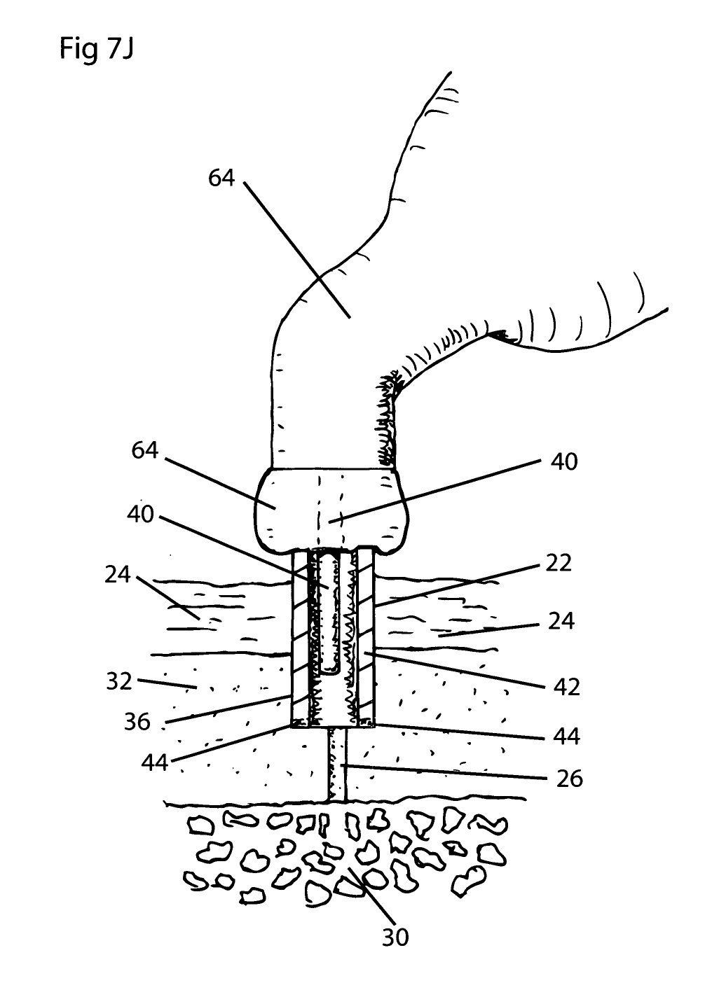

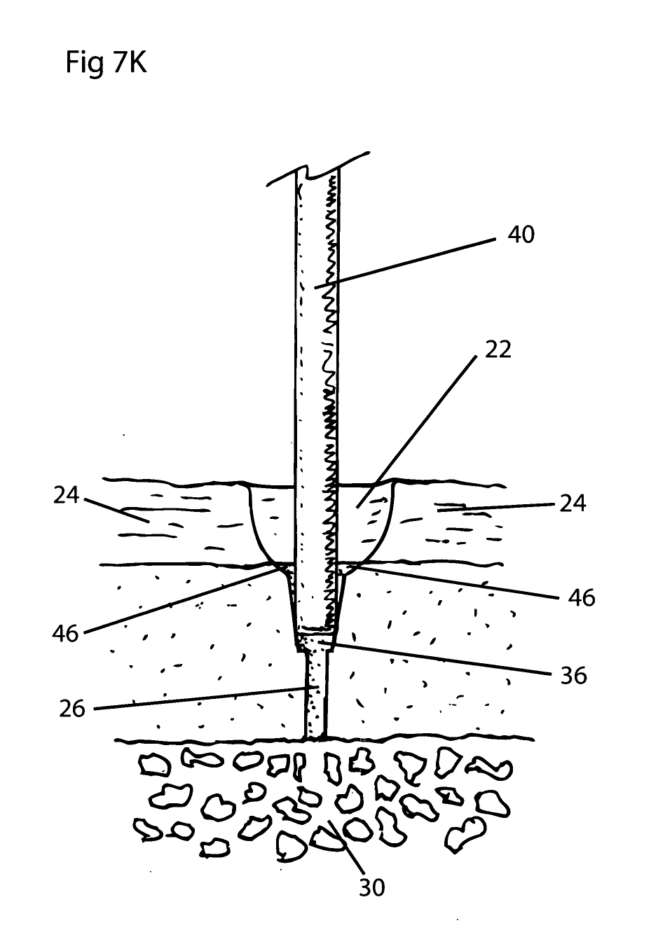

FIGS. 7A-7K show various methods of obtaining a fluid-tight injector seal against proximal bone 32 or gingiva 24.

FIG. 7A shows an injector 40 having a diameter that is greater than the diameter of gingival access 22 and access seat 36. A suitable diameter for such an injector 40 is 17 gauge with ID 1.067 and OD 1.473 mm. Injector 40 compresses the surface of gingiva 24 about gingival access 22 to create a fluid-tight seal. As injector 40 compresses gingiva 24, injector 40 forms a substantial indentation in the surface of gingiva 24. The diameter of access seat 36 is sufficient to prevent gingiva 24 from slumping inward and interfering with the fluid flow. When a fluid is injected from injector 40, the seal contains the fluid, and directs it into access seat 36, into conduit 26, and into cancellous bone 30.

FIG. 7B shows an injector 40 having a diameter that is smaller than the diameter of access seat 36, such that injector 40 does not sealingly engage access seat 36. Injector 40 functions to facilitate locating gingival access 22, and to stabilize the position of hub 64 over gingival access 22 during injection.

For a 0.908 mm diameter access seat 36, suitable injectors 40 include 22 gauge (OD 0.7176 mm), 23 gauge, and so on. The diameters of gingival access 22 and access seat 36 are sufficiently large to facilitate rapid location by the user and rapid insertion of injector 40. Injector 40 protrudes distally from a large diameter hub 64. The diameter of hub 64 is greater than the diameter of gingival access 22. Hub 64 is sealingly pressed against gingiva 24, thereby compressing gingiva 24 to create a fluid-tight seal. A suitable diameter for the distal end of hub 64 is 2.5 mm.

Injector 40 has a sufficiently shortened length such that injector 40 does not interfere with the movement of hub 64 toward gingiva 24 by inadvertently contacting the end-stop of access seat 36 when hub 64 sealingly compresses gingiva 24. The distance injector 40 protrudes from hub 64 is less than the combined depth of gingival access 22 and access seat 36. Injector 40 has a sufficiently short length such that interfering contact with the access seat 36 end-stop is unlikely even with a shallow gingival access 22. An injector 40 having a length in the range of 2 to 3 mm is unlikely to interfere with a 3 mm depth access seat 36 end-stop even when compressing a gingiva 24 having a depth of only 1 mm.

It is preferred that hub 64 has an elastomeric distal end encompassing injector 40 to facilitate forming a fluid-tight seal with gingiva 24. The elastomeric distal end may be integral with hub 64, may be adhered to hub 64, may comprise an elastomeric stopper impaled by injector 40, and so on. When pressed against the surface of gingiva 24 or proximal bone 32, the elastomeric distal end conforms to surface irregularities to form a fluid-tight seal with hub 64. Further, a dissolvable gel or gum may be located on the distal end of hub 64 for enhancing the seal against gingiva 24.

It is preferred that the distal end portion of hub 64 is substantially angled with respect to the long axis of a syringe to which hub 64 is connected, to facilitate convenient access to confined workspaces. It is preferred that at least a portion of the distal end of hub 64 is transparent to facilitate user visualization of injector 40 as it is inserted into gingival access 22. It is preferred that the distal end of hub 64 has a substantially flattened configuration. However, the distal end of hub 64 may be conical, concave, convex, and so on. When a fluid is injected from injector 40, the seal contains the fluid, and directs it into access seat 36, into conduit 26, and into cancellous bone 30.

FIG. 7C shows an injector 40 having a diameter that is smaller than the diameter of conduit 26. Injector 40 protrudes distally from a hub 64. The small diameter of injector 40 provides a clearance with conduit 26, and minimizes interference to insertion. A funnel-shaped access seat 36 further minimizes bony interferences for inserting injector 40 into conduit 26. The diameter of injector 40 is smaller than conduit 26, and so is too small to sealingly engage conduit 26. However, the diameter of hub 64 is greater than the diameter of gingival access 22. Hub 64 is shown sealingly compressing gingiva 24 to form a fluid-tight seal.

Hub 64 has an elastomeric distal end encompassing injector 40 for forming a fluid-tight seal with gingiva 24. The elastomeric distal end is several millimeters in length. As such, the elastomeric portion is readily bent to improve user access in a restricted site. Injector 40 is shown connecting through the elastomeric portion to a non-elastic portion of hub 64. As such, injector 40 is bent to the degree that the elastomeric portion is bent. However, the elastomeric portion may have a lumen connected to injector 40 so injector 40 does not connect to a non-elastic portion of hub 64, all of hub 64 may be elastomeric, the elastomeric portion may be disconnected from hub 64 and wherein the elastomeric portion comprises a stopper about injector 40, and so on. When a fluid is injected from injector 40, the seal contains the fluid, and directs it into access seat 36, into conduit 26, and into cancellous bone 30.

Suitable diameters for such an injector 40 include 28 gauge with OD 0.3366 mm, 29 gauge, 30 gauge, and so on. Suitable lengths for such an injector 40 to extend distally from hub 64 are in the range of 1-8 mm, but more preferably 4 mm. Alternatively, a small diameter injector 40 may be similarly inserted into a conduit 26 having no access seat 36 enlargement of proximal bone 32, wherein hub 64 is sealingly pressed against gingiva 24.

FIG. 7D shows an injector 40 having a diameter that is smaller than the diameter of access seat 36. For a 0.908 mm diameter access seat 36, suitable diameters for such an injector 40 include 21 gauge 0.8192 mm, 22 gauge, 23 gauge and so on. The larger diameter of access seat 36 facilitates rapid location of access seat 36 by the user and rapid insertion of injector 40. Injector 40 protrudes a short distance distally from hub 64, such as 3 mm. The diameter of hub 64 is greater than the diameter of access seat 36. A small sleeve 62, shown in cross-section, is shown inserted into gingival access 22, wherein the distal end of small sleeve 62 contacts the surface of proximal bone 32, or periosteum cover thereof. The small sleeve 62 is comprised of an elastomeric material. It is preferred that hub 64 has an elastomeric distal end encompassing injector 40 to facilitate forming a fluid-tight seal with gingiva 24 or with small sleeve 62.

Hub 64 is sealingly pressed against the proximal end of small sleeve 62 to form a fluid-tight seal therewith. Further, pressure is transferred from hub 64 to the distal end of small sleeve 62, wherein the pressure sealingly presses the distal end of small sleeve 62 against the surface of proximal bone 32 to form a fluid-tight seal therewith. It is not critical that injector 40 sealingly engage access seat 36 to form a fluid-tight seal therewith.

In a variation of the method, injector 40 is sealingly inserted into small sleeve 62 such that a fluid-tight seal is formed between the adjacent parallel cylindrical walls of injector 40 and small sleeve 62. As such, injector 40 is tightly and sealingly inserted into small sleeve 62. In a further variation of the method, an elastomeric stopper may be impaled upon injector 40 prior to insertion into gingival access 22. As such, the stopper functions with injector 40 in a manner similar to small sleeve 62, and would appear identically to sleeve 62 as depicted in FIG. 7D. Pressure on stopper small sleeve 62 forms a fluid-tight seal between hub 64 and proximal bone 32. When a fluid is injected from injector 40, the seal contains the fluid, and directs it into access seat 36, into conduit 26, and into cancellous bone 30.

FIG. 7E shows gingiva 24 sealingly interposed between inserted injector 40 and the surfaces of access seat 36. The injector 400D is smaller than the diameter of access seat 36. For a 0.908 mm diameter access seat 36, a suitable injector 40 includes 22 gauge 0.7176 mm OD, 23 gauge, and so on. Prior to the insertion of injector 40, adjacent gingiva 24 tends to slump across the orifice of access seat 36. During insertion, the tip of injector 40 engages and pushes the slumping gingiva 24 into access seat 36, such that gingiva 24 is sealingly interposed between inserted injector 40 and the surfaces of access seat 36. Injector 40 sealingly is pressed against gingiva 24, and the pressed gingiva 24 is in turn sealingly pressed against access seat 36. As such, gingiva 24 forms a fluid-tight seal with an injector 40 having an OD that is smaller than the diameter of access seat 36. When a fluid is injected from injector 40, the seal contains the fluid, and directs it into access seat 36, into conduit 26, and into cancellous bone 30.

FIG. 7F shows an injector 40 having a diameter that is greater than the diameter of access seat 36. Gingival access 22 has been enlarged to a diameter greater than access seat 36, such as by a sleeve 42. A fluid-tight seal is formed by pressing the tip of injector 40 against the uncut proximal bone 32 surface area surrounding access seat 36, or against the periosteum thereover. When a fluid is injected from injector 40, the seal contains the fluid, and directs it into conduit 26, and into cancellous bone 30. A suitable diameter for such an injector 40 would be 17 gauge, with OD 1.473 mm and ID 1.067 mm. Alternatively, a fluid-tight seal may be formed by an injector 40 having a diameter slightly larger than access seat 36 sealingly pressing against the orifice edge of access seat 36, or bevel 46 thereof. Further, a fluid-tight seal may be formed by pressing the tip of an injector 40 against the uncut proximal bone 32 surface area, or periosteum thereover, that encompasses a conduit 26 having no access seat 36. Yet further, a fluid-tight seal may be formed by injector 40 sidewalls pressing laterally against the cylindrical sidewalls of gingival access 22 within gingiva 24.

FIG. 7G shows an injector 40 sealingly inserted into proximal conduit 26, wherein the injector 40 diameter is the same or slightly larger than the diameter of conduit 26. Injector 40 is readily inserted through gingival access 22 within gingiva 24. Injector 40 is inserted into proximal conduit 26 with pressure, wherein the tip and sidewalls of injector 40 press against and sealingly engage the sidewalls of conduit 26 to form a fluid-tight seal. The injector 400D is the same as, or slightly larger than, the diameter of the perforator 12 which formed conduit 26. For example, tightly inserting a 25 gauge 0.5144 mm OD injector 40 into a proximal conduit 26 formed by a 0.4636 mm diameter perforator 12 forms a fluid-tight seal. As such, when fluid is injected from injector 40, the seal contains the fluid and directs the fluid from proximal conduit 26, through distal conduit 26, and into cancellous bone 30. It is preferred that injector 40 is not inserted into distal conduit 26 to avoid substantial interferences to insertion. The funnel-shaped access seat 36 shown facilitates smooth insertion of injector 40 into conduit 26.

FIG. 7H shows an injector 40 sealingly engaged into proximal conduit 26 in proximal bone 32. Gingiva 24 has no gingival access 22 enlargement, and proximal conduit 26 has no access seat 36 enlargement. Injector 40 has a diameter that is the same or slightly larger than the diameter of conduit 26. Injector 40 is inserted into proximal conduit 26 with pressure such that the tip and sidewalls of injector 40 press against and sealingly engage the sidewalls of conduit 26 to form a fluid-tight seal. For example, tightly inserting a 25 gauge 0.5144 mm OD injector 40 into proximal conduit 26 having a 0.4636 mm diameter can form a fluid-tight seal. It is preferred that injector 40 is not inserted into distal conduit 26 to avoid substantial interferences to insertion. As such, when fluid is injected from injector 40, the seal between injector 40 and proximal conduit 26 contains the fluid and directs the fluid into distal conduit 26, and into cancellous bone 30.

FIG. 7I shows an access seat 36 where countersink 14 has perforated entirely through cortical bone 28. Access seat 36 is essentially extended entirely through cortical bone 28. No access seat 36 end-stop remains and conduit 26 has been obliterated. An injector 40 is inserted through gingival access 22 within gingiva 24, and sealingly engaged into extended access seat 36 within proximal bone 32. Injector 40 has a diameter that is the same or slightly larger than the diameter of access seat 36. Injector 40 is inserted into the proximal portion of extended access seat 36 with pressure such that the tip and sidewalls of injector 40 press against and sealingly engage the sidewalls of access seat 36 to form a fluid-tight seal. For example, tightly inserting a 19 gauge 1.067 mm OD injector 40 into proximal extended access seat 36 having a 0.9081 mm diameter can form a fluid-tight seal. It is preferred that injector 40 is not inserted into distally extended access seat 36 to avoid substantial interferences to insertion. As such, when fluid is injected from injector 40, the seal between injector 40 and proximally extended access seat 36 contains the fluid and directs the fluid into distally extended access seat 36, and into cancellous bone 30.

FIG. 7J shows a sleeve 42 having enlarged a gingival access 22 within gingiva 24, and an access seat 36 into proximal bone 32 with burs 44. Sleeve 44 has penetrated into proximal bone 32 somewhat beyond the depth of gingival access 24, thereby enhancing the stability and retention of sleeve 42 in proximal bone 32. The width of the bur 44 cutting-surface has the same width as the sleeve 42 sidewall thickness. As such, sleeve 42 has no ring 48 depth-stop. The length of sleeve 42 in FIG. 7J is the same as the length of countersink 14. As such, access seat 36 is not bored to a further depth into proximal bone 32 than countersink 14. Sleeve 42 remains inserted into enlarged access seat 36 after removal of the remainder of bit 10. As such, the proximal portion of sleeve 42 protrudes from proximal bone 32 for maintenance of gingival access 22 patency. Injector 40 is loosely inserted into sleeve 42. Hub 64 is sealingly pressed against the proximal end of sleeve 42. As such, when fluid is injected from injector 40, the seal between hub 64 and sleeve 42 contains the fluid and directs the fluid into distally extended access seat 36, into conduit 26, and into cancellous bone 30.

FIG. 7K shows an injector 40 sealingly pressed into a tapered access seat 36. The tapered access seat 36 has been formed by a tapered countersink 14, such as shown in FIG. 5B. Tapered access seat 36 has a 0.04 taper. For example, for a 0.04 tapered access seat 36 having an orifice diameter of 0.908 mm, the diameter at a depth of 3 mm is 0.799 mm. As such, a 21 gauge injector 40 having an OD 0.8192 mm is readily insertable into the 0.9081 mm orifice of access seat 36. When such an injector 40 is inserted to near the end-stop of tapered access seat 36, injector 40 engages and sealingly presses against the sidewalls of tapered access seat 36. The gradual taper of tapered access seat 36 facilitates a smooth, ever-tightening, insertion of injector 40 such that a fluid-tight seal is formed.

For a tapered access seat 36 having a 0.908 mm diameter orifice, a blunt 21 gauge injector 40 having an OD 0.8192 mm is selected. User quickly locates the wide beveled gingival access 22. Gingiva 24 does not slump over access seat 36. Injector 40 is readily inserted through beveled gingival access 22. Bevel 46 readily centers injector 40 into the 0.9081 mm diameter orifice of access seat 36.

Injector 40 freely slides further into deeper portions of access seat 36 where the diameter is reduced. When injector 40 reaches the portion of access seat 36 where the access seat 36 diameter is the same or smaller than 0.8192 mm, injector 40 engages and binds against the sidewalls of access seat 36. Injector 40 is sealingly pressed against the sidewalls of access seat 36 such that a fluid-tight seal is formed. As such, fluid from injector 40 is contained by the seal, and is directed through conduit 26 and into cancellous bone 30.

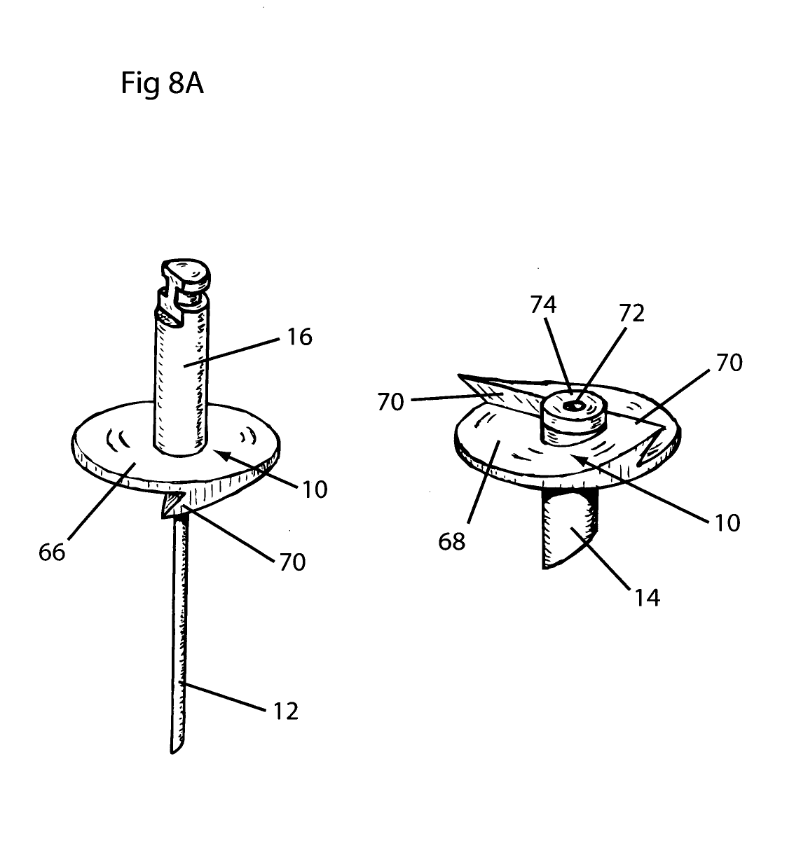

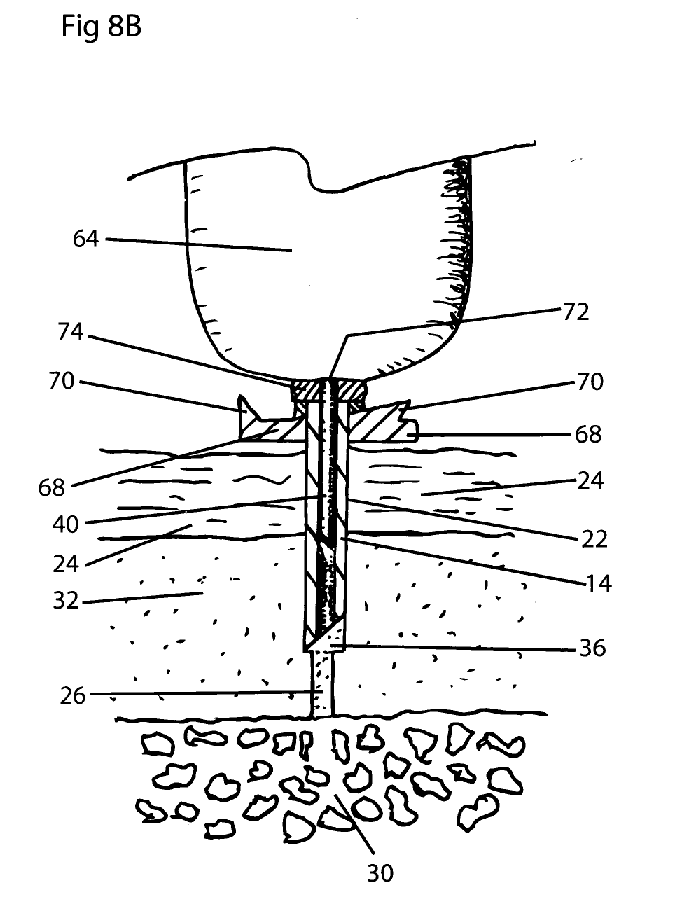

FIG. 8A shows a bit 10 having a detachable countersink 14. Bit 10 is disassembled. Shank 16 has a shank disc 66 and countersink 14 has a mated countersink disc 68. Shank discs 66 and countersink disc 68 each have protruding blades 70. Blades 70 have edges overhanging undercuts. When the discs are mated together, the blades 70 mutually engage. When forcefully rotated, the blade 70 undercuts detachably lock together. In some embodiments, blades 70 include interlocks that are perpendicular with respect to the long axis of bit 10, for additional locking retention.

At the center of countersink disc 68, a cylinder securely encompasses and grips the countersink 14 metal needle. As such, countersink 14 does not break free from countersink disc 68 during forceful rotation. The cylinder has a central lumen 72 that is continuous with the lumen of countersink 14. Lumen 72 has a diameter that provides a degree of resistance to movement of perforator 12, such that perforator 12 and countersink 14 are not likely to separate inadvertently. The cylinder may further comprise a lumen of an elastomeric stopper 74 set in the cylinder, such that perforator 12 elastically slides through lumen 72. It is preferred that countersink 14 extends 4 mm distally from countersink disc 68.

When boring access seat 36, blades 70 lock countersink 14 to shank 16. When shank 16 stops rotating, blades 70 are readily releasable from the undercuts. Shank 16 and perforator 12 are detachable from countersink 14, and are readily withdrawn from the injection site.

For removal of countersink 14 from access seat 36, perforator 12 is completely re-inserted through lumen 72 and countersink 14 until blades 70 interlock. Bit 10 is rotated to lockingly engage opposing sets of blades 70, and to reduce starting friction of perforator 12 and countersink 14 with proximal bone 32. As bit 10 is withdrawn from the site, interlocking blades 70 lift countersink 14 from proximal bone 32. When countersink 14 is withdrawn from the site, the rotation force diminishes such that blades 70 will not retentively interlock. Countersink 14 remains retained to perforator 12 and shank 16 by friction with stopper 74.

FIG. 8B shows a detachable countersink 14 detached from shank 16 after boring a gingival access 22 and an access seat 36 in proximal bone 32. Countersink 14 remains retentively and sealingly inserted into access seat 36. Countersink disc 68 rests near or against the gingiva 24 surface. Blades 70 protrude from the surface of countersink disc 68. Injector 40 is inserted through lumen 72 such that the injector 40 tip is within the lumen of countersink 14. Hub 64 is sealingly pressed against stopper 74. As such, fluid injected from injector 40 is contained by the fluid-tight seal, and is directed through countersink 14, through conduit 26, and into cancellous bone 30.

From the description above, a number of advantages of the intraosseous bit become evident:
(a) a cortical bone conduit and injector access seat are rapidly borable
(b) an injector is rapidly insertable into an access seat
(c) an access seat forms a fluid-tight seal with an injector
(d) stresses exerted on the bone bit are minimized
(e) reinjection through a bony conduit is convenient
(f) risk of neglecting to remove sharps from mouth is decreased
(g) the system facilitates patient acceptance

### Operation—FIGS. 1-7

By using the intraosseous bit 10 of the invention, it is now possible, surprisingly, to rapidly perforate cortical bone 28, and rapidly sealingly seat an injector 40 against proximal bone 32 or gingiva 24 for injection into cancellous bone 30. The process offers the advantage that a user can now inject fluid into cancellous bone 30 without substantial fluid leakage into the mouth.

### Example A

In a further embodiment of the invention, implementation of the process begins with pre-anesthetising gingiva 24 over an intraosseous injection site. A bit 10 without sleeve 42, as shown in FIG. 1, is inserted into handpiece 38. Perforator 12 extends 5 mm distally beyond countersink 14, and has a diameter of 0.4636 mm, and countersink 140D is 0.9081 mm. A first mark 18 has the original coloration of countersink 14 over the distal 3 mm. A second mark 18 is black from 3 mm from the distal end of countersink 14 to 6 mm from the distal end.

Bit 10 is rotated and perforator 12 is pressed against gingiva 24. Perforator 12 begins to bore a gingival access 22 through gingiva 24 until proximal bone 32 is contacted. Small diameter perforator 12 rapidly bores proximal conduit 26 into proximal bone 32. Countersink 14 enlarges gingival access 22 and advances until it contacts proximal bone 32. The junction between the first and second marks 18 is even with the surface of gingiva 24, indicating that gingiva 24 is 3 mm in depth over proximal bone 32. When countersink 14 contacts proximal bone 32, perforator 12 has drilled conduit 26 to a depth of 5 mm. Perforator 12 is at a depth of 5 mm into proximal bone 32, plus a 3 mm depth in gingiva 24, so that perforator 12 is bored to a total depth of 8 mm below the surface of gingiva 24.

The patient's cortical bone 28 happens to be unusually hard and thick, and perforator 12 inadvertently fractures. The fracture occurs at nick 20 because it comprises the narrowest diameter of perforator 12. The remainder of bit 10 is removed from the injection site by sliding countersink 14 off the fractured distal portion of perforator 12. Fractured perforator 12 is 17 mm in length from the distal end to nick 20. When perforator 12 is bored 8 mm into gingiva 24 and proximal bone 32, then 9 mm of perforator 12 remains protruding above gingiva 24. User extracts fractured perforator 12 with a hemostat. A new bit 10 is loaded into handpiece 38, and perforator 12 of the new bit 10 is inserted to the current bore depth of conduit 26.

Countersink 14 is advanced into proximal bone 32 until the junction between the second and third mark 18 is even with the surface of gingiva 24, signaling the user that access seat 36 has been bored to a depth of 3 mm into proximal bone 32. Countersink 14 bores access seat 36 in a short time because the depth is only 3 mm, and the diameter is 0.908 mm. Countersink 14 is able to cut access seat 36 in a short time period because the cutting tip remains very sharp when cutting to a depth of only 3 millimeters into proximal bone 32, the sidewall friction is minimal at a depth of 3 mm, and the total volume of bone removed is minimal for a 0.908 mm diameter access seat 36. The configuration of perforator 12 and countersink 14 minimize the total work required to bore both conduit 26 and access seat 36.

As countersink 14 bores access seat 36 to a depth of 3 mm, perforator 12 simultaneously bores distal conduit 26 an additional 3 mm deeper, penetrating through cortical bone 28 and into cancellous bone 30, as shown in FIG. 2. Boring is complete, and bit 10 is removed from the mouth.

User quickly locates gingival access 22 because the diameter is sufficiently large. An injector 40 having an OD of 0.7176 mm is inserted into gingival access 22. However, gingiva 24 is slumping into, and partly occluding the lumen of, gingival access 22. As the tip of injector 40 moves into access seat 36, it impinges on the slumping gingiva 24. Injector 40 thereby pushes the slumping gingiva 24 ahead of the injector 40 tip, and partly into access seat 36. As the tip of injector 40 is inserted beyond the orifice of access seat 36, the tip pulls gingiva 24 further into access seat 36. As injector 40 bottoms in access seat 36, a thin layer of gingiva 24 is forcefully compressed circumferentially between injector 40 and the walls of access seat 36. The forceful compression of gingiva 24 between injector 40 and the walls of access seat 36 forms a fluid-tight seal, as shown in FIG. 7E.

User activates a fluid-containing syringe associated with injector 40 so that pressure is applied to the fluid, and pressurized fluid is ejected from injector 40. Leakage from between injector 40 and access seat 36 is minimized because interposed gingiva 24 is sealingly compressed. The seal between injector 40, gingiva 24, and access seat 36, is substantially leak-tight and able to contain the pressurized fluid from injector 40. The seal thereby maintains the fluid pressure, such that the pressurized fluid from injector 40 is directed through conduit 26 and into the spaces of cancellous bone 30. After sufficient volume of fluid has been injected into cancellous bone 30, injector 40 is withdrawn from the mouth. The initial intraosseous injection is complete.

Thirty minute after the initial intraosseous injection, user finds that additional fluid is required for injection in cancellous bone 30. The intraosseous injection process is repeated by readily relocating gingival access 22. Gingiva 24 has again slumped into the insertion path of injector 40. As injector 40 is reinserted into gingival access 22 and access seat 36, gingiva 24 is pushed into access seat 36. Injector 40 and gingiva 24 again form a seal against the surfaces of access seat 36. User re-injects fluid through conduit 26 into cancellous bone 30. Injector 40 is withdrawn from the mouth, completing the intraosseous re-injection.

### Example B

User pre-anesthetises gingiva 24 over an intraosseous injection site. The gingiva over the site is deep and loose, so user selects a bit 10 having a sleeve 42 over large axle 50, as shown in FIG. 6A. The large axle 50 diameter is 1.27 mm, and the ID of sleeve 42 is 1.37 mm.

The length of sleeve 42 extends 6 mm from shank 16. Sleeve 42 leaves the distalmost 3 mm of countersink 14 protruding uncovered. The distal half of sleeve 42 has a 3 mm long dark sleeve marking 60, and the proximal half has a 3 mm non-colored sleeve marking 60. Sleeve 42 also has a gel coating for retention at the site. Countersink 14 has an OD of 0.9081 mm, and is tapered, as shown in FIG. 4C. Perforator 12 is 26 gauge having a 0.4636 mm diameter, and extends 5 mm distally from countersink 14.

Bit 10 is inserted into handpiece 38. Bit 10 is rotated and perforator 12 is pressed against gingiva 24 to bore a gingival access 22 to proximal bone 32. Perforator 12 contacts proximal bone 32 at the same time that countersink 14 contacts gingiva 24, revealing that gingiva 24 is approximately 5 mm in depth at the site. As perforator 12 begins boring conduit 26 into proximal bone 32, countersink 14 enlarges gingival access 22. After perforator 12 has advanced 3 mm into proximal bone 32, sleeve 42 comes into contact with the surface of gingiva 24. As perforator 12 advances further into cortical bone 28, countersink 14 and sleeve 42 enlarge gingival access 22. Two sleeve offsets 56 on the proximal end of sleeve 42 engage two shank offsets 50 on the distal end of shank 16 to rotate sleeve 42. Two sleeve offsets 56 on the distal edge of sleeve 42 cut gingiva 24 to enlarge gingival access 22. Perforator 12 rapidly bores conduit 26 due to the small diameter.

Countersink 14 advances through gingiva 24 until it contacts proximal bone 32. With countersink 14 resting on the surface of proximal bone 32, user notes that 1 mm of the dark sleeve marking 60 on the distal half of sleeve 42 still shows above the surface of gingiva 24. Since the dark sleeve marking 60 has a length of 3 mm, user concludes that sleeve 42 has penetrated into gingiva 24 to a depth of 2 mm. Because countersink 14 extends 3 mm beyond sleeve 42, user concludes that countersink 14 is at a depth of 5 mm into gingiva 24. Since countersink 14 is resting in contact with the surface of proximal bone 32, user confirms that the depth of gingiva 24 over the site is 5 mm. When countersink 14 contacts proximal bone 32 surface, perforator 12 has bored into cortical bone 26 to a depth of 5 mm.

As countersink 14 advances and penetrates into proximal bone 32, sleeve 42 cuts into gingiva 24, thereby enlarging gingival access 22. After countersink 14 has advanced 3 mm into proximal bone 32, sleeve 42 comes into contact with the surface of proximal bone 32. The distal end of sleeve 42 does not cut proximal bone 32, but only spins upon the surface of proximal bone 32. The proximal end of sleeve 42 is engaged by the distal end of shank 16, and pressed toward proximal bone 32. Sleeve 42 thereby stops the penetration of countersink 14 into proximal bone 32 at 3 mm. As such, sleeve 42 limits the depth of access seat 36 into proximal bone 32, and functions as a countersink 14 depth-stop, as shown in FIG. 6A. Perforator 12 has bored a 0.46 mm diameter conduit 26 entirely through cortical bone 28 and into cancellous bone 30. The tip of perforator 12 is 8 mm distal from the surface of proximal bone 32.

The sleeve 42 gel coating has partly dissolved in fluids present in gingival access 22, and has become sticky. Sticky sleeve 42 develops a degree of retentive adhesion to gingival access 22. As the remainder of bit 10 is withdrawn from the site, sleeve 42 retentively adheres to gingival access 22 such that sleeve 42 slides off large axle 50. As such, sleeve 42 remains inserted in gingival access 22 in contact with proximal bone 32, as shown in FIG. 6B. The proximal 1 mm of sleeve 42 protrudes from the 5 mm-deep gingival access 22.

With sleeve 42 maintaining the patency of gingival access 22, a 25 gauge 0.5144 mm OD injector 40 has ample clearance with the 1.372 mm ID of sleeve 42 to be readily inserted down to proximal bone 32. Injector 40 is conveniently and rapidly centered into the 0.9081 mm diameter orifice of access seat 36. Injector 40 is readily inserted through tapered access seat 36. The access seat 36 taper smoothly deflects and centers injector 40 into conduit 26 without substantial interference.

The 0.5144 mm OD injector 40 encounters firm resistance as the tip gradually wedges into the tapered orifice of 0.46 mm diameter conduit 26. Injector 40 is sealingly seated with moderate pressure into proximal conduit 26, such that a fluid-tight seal is formed, as shown in FIG. 7D.

User activates a fluid-containing syringe connected to injector 40 so that pressure is applied to the fluid, and pressurized fluid is ejected from injector 40. Leakage from between injector 40 tip and proximal conduit 26 is minimized because injector 40 tip is sealingly seated into conduit 26. The seal between injector 40 and proximal conduit 26 is substantially leak-tight and is able to contain the pressurized fluid from injector 40, and thereby maintains the fluid pressure. As such, the pressurized fluid from injector 40 is directed into distal conduit 26 and into cancellous bone 30.

After sufficient volume of fluid has been injected into cancellous bone 30, injector 40 is readily withdrawn from the site without dislodging sleeve 42. The initial intraosseous injection is complete. Sleeve 42 is allowed to remain in gingival access 22 to keep gingival access 22 patent for possible re-injection. The patient is accepting of sleeve 42 because the use is comfortable, sleeve 42 is unobtrusive, and no sharp parts remain in the mouth except during direct use.

Thirty minute after the initial intraosseous injection, user finds that additional fluid is required for injection in cancellous bone 30. The intraosseous injection process is readily repeated by reinserting injector 40 into sleeve 42 and access seat 36. User exerts pressure to sealingly seat injector 40 into proximal conduit 26, and re-injects fluid through distal conduit 26 and into cancellous bone 30. Injector 40 is withdrawn from the mouth, completing the intraosseous re-injection.

When sleeve 42 is no longer needed, it is conveniently removed by grasping with cotton pliers and withdrawing from gingival access 22. The mild adhesion between gingival access 22 and sleeve 42 releases sleeve 42, and the procedure is completed.

### Example C

In a further embodiment of the invention, implementation of the process begins with pre-anesthetising gingiva 24 over an intraosseous injection site. A bit 10 having an affixed sleeve 42 is inserted into handpiece 38. Sleeve 42 has a multiplicity of burs 44, a ring 48, and a beveled outer-wall cutter for beveling gingival access 22, as shown in FIG. 5B. Countersink 14 is slightly tapered, having a 0.9091 mm diameter proximally to sleeve 42, and tapering to a 0.7995 mm distal diameter. Perforator 12 extends 5 mm distally beyond countersink 14.

Perforator 12 bores a gingival access 22 and a conduit 26 into proximal bone 32. Countersink 14 enlarges gingival access 22, and begins to bore a tapered access seat 36. As countersink 14 bores access seat 36, bur 44 and the beveled outer-wall cutter of sleeve 42 bevels gingival access 22. As access seat 36 is bored to a depth of 3 mm, bur 44 forms a bevel into proximal bone 32 at the orifice of access seat 36. Once access seat 36 reaches a depth of 3 mm, ring 48 contacts the surface of proximal bone 32. Ring 48 does not cut proximal bone 32, and therefore stops countersink 14 from boring access seat 36 deeper than 3 mm into proximal bone 32. As countersink 14 bores access seat 36 to a depth of 3 mm, perforator 12 penetrates through cortical bone 28 into cancellous bone 30. Boring is complete, and bit 10 is removed from the mouth.

A blunt 21 gauge injector 40 having an OD 0.8192 mm is selected. User quickly locates the wide beveled gingival access 22. Gingiva 24 does not slump over access seat 36. Injector 40 is readily inserted through beveled gingival access 22. Bevel 46 readily centers injector 40 into the 0.9081 mm diameter orifice of access seat 36.

Injector 40 freely slides further into deeper portions of access seat 36 where the diameter is reduced. When injector 40 reaches the portion of access seat 36 where the access seat 36 diameter is the same or smaller than 0.8192 mm, injector 40 engages and binds against the sidewalls of access seat 36. Injector 40 is sealingly pressed against the sidewalls of tapered access seat 36 such that a fluid-tight seal is formed, as shown in FIG. 7K.

User activates a fluid-containing syringe connected to injector 40 such that pressurized fluid is ejected from injector 40. Leakage from between injector 40 tip and access seat 36 is minimized because injector 40 tip is sealingly seated into tapered access seat 36. The seal between injector 40 and access seat 36 is substantially leak-tight and is able to contain the pressurized fluid from injector 40, and thereby maintains the fluid pressure. As such, the pressurized fluid from injector 40 is directed through conduit 26 and into the spaces of cancellous bone 30. After sufficient volume of fluid has been injected into cancellous bone 30, injector 40 is entirely withdrawn from the mouth. The initial intraosseous injection is complete.

Thirty minute after the initial intraosseous injection, user finds that additional fluid is required for injection in cancellous bone 30. The intraosseous injection process is readily repeated by relocating the wide, beveled gingival access 22, and reinserting injector 40 into gingival access 22 and access seat 36. User presses injector 40 into the slightly tapered access seat 36 until it sealingly seats against the surfaces of the restricted-diameter depths of access seat 36. Fluid is re-injected from injector 40 through conduit 26, and into cancellous bone 30. Injector 40 is withdrawn from the mouth, completing the intraosseous re-injection.

### Example D

In a further embodiment of the invention, implementation of the process begins with pre-anesthetising gingiva 24 over a restricted access intraosseous injection site. A bit 10 is selected having an affixed sleeve 42 having a rounded distal edge, as shown in FIG. 5C. The distalmost 0.5 mm of sleeve 42 is coated with 30 μm diamonds. A 2 mm length band of 100 μm medium grit abrasive is located immediately proximal to the fine grit abrasive of the distal end. The diameter of perforator 12 is 0.4636 mm, countersink 140D is 0.9081 mm, and sleeve 420D is 1.473 mm. Countersink 14 protrudes 3 mm distally from sleeve 42. Bit 10 is inserted into handpiece 38.

Perforator 12 bores a gingival access 22 and a conduit 26 into proximal bone 32. Countersink 14 enlarges gingival access 22, and begins to bore an access seat 36. As countersink 14 rapidly bores access seat 36, the abrasive distal end of sleeve 42 enlarges gingival access 22.

As countersink 14 nears a depth of 3 mm when boring access seat 36, the abrasive distal end of sleeve 42 contacts the thin, sharp edge of the access seat 36 orifice. The 30 μm diamonds rapidly remove a small volume of proximal bone 32 from the orifice of access seat 36 to form bevel 46. The 2 mm long band of 100 μm diamonds readily widens gingival access 22 away from the orifice of access seat 36. As such, gingiva 24 is less likely to slump into access seat 36, or interfere with the insertion of injector 40.

As the width of bevel 46 nears 0.2 mm, the low cutting efficiency of the 30 μm diamonds causes substantial cutting resistance. As such, further insertion of bit 10 into proximal bone 32 is substantially slowed. The bit 10 insertion resistance provides tactile feedback for the user, signaling that bevel 46 has been bored to an adequate width, and countersink 14 has bored access seat 36 to 3 mm. Access seat 36 is 3 mm in depth, and perforator 12 has penetrated through cortical bone 28 into cancellous bone 30. Boring is complete, and bit 10 is removed from the mouth.

A 0.4128 mm OD injector 40 is selected because the diameter is smaller than the 0.464 mm diameter of conduit 26. A hub 64 has an elastomeric distal end portion encompassing injector 40. The elastomeric distal end portion extends 8 mm distally from a non-elastomeric portion of hub 64. Injector 40 protrudes 5 mm distally from the elastomeric distal end of hub 64. Injector 40 connects through the elastomeric portion to the non-elastic portion of hub 64.

The user bends the elastomeric end portion of hub 64 and injector 40 about 60° to improve access to the restricted site, as shown in FIG. 7C. The elastomeric portion of hub 64 is sufficiently pliable to permit the bend angle to remain.

User quickly locates widened gingival access 22. Gingiva 24 does not slump over access seat 36. Injector 40 is readily inserted through widened gingival access 22. Bevel 46 readily centers injector 40 into the 0.9081 mm diameter orifice of access seat 36. The 0.4128 mm OD injector 40 has sufficient clearance with 0.464 mm conduit 26 for insertion without substantial interference. However, injector 40 does not sealingly engage conduit 26.

Injector 40 is inserted into conduit 26 until the elastomeric distal end of hub 64 seats against gingiva 24 about gingival access 22. The diameter of hub 64 is greater than the diameter of gingival access 22. The elastomeric end of hub 64 is sealingly pressed against gingiva 24 to form a fluid-tight seal. The elastomeric distal end of hub 64 conforms to the irregular surface of gingiva 24 to facilitate forming a fluid-tight seal.

User activates a fluid-containing syringe connected to injector 40 such that pressurized fluid is ejected from injector 40. Leakage from between injector 40 tip and access seat 36 is minimized because the elastomeric end of hub 64 is sealingly pressed against gingiva 24 to form a substantially fluid-tight seal. The seal between hub 64 and gingiva 24 is able to contain the pressurized fluid from injector 40, and thereby maintains the fluid pressure. As such, the pressurized fluid from injector 40 is directed through conduit 26 and into the spaces of cancellous bone 30. After sufficient volume of fluid has been injected into cancellous bone 30, injector 40 is entirely withdrawn from the mouth. The initial intraosseous injection is complete.

Thirty minutes after the initial intraosseous injection, user finds that additional fluid is required for injection in cancellous bone 30. The intraosseous injection process is readily repeated by relocating the widened gingival access 22, and reinserting injector 40 into gingival access 22, access seat 36, and proximal conduit 26. User sealingly seats the elastomeric distal end of hub 64 against gingiva 24. Fluid is re-injected from injector 40 through conduit 26, and into cancellous bone 30. Injector 40 is withdrawn from the mouth, completing the intraosseous re-injection.

### Example E

User pre-anesthetises gingiva 24 over a confined posterior intraosseous injection site. A bit 10 having a 20 gauge 0.9081 mm diameter countersink 14 for boring an access seat 36, such as shown in FIG. 2, is inserted into handpiece 38. No sleeve 42 or small sleeve 62 is present. Perforator 12 is 26 gauge. Bit 10 bores gingival access 22, a 3 mm depth access seat 36, and conduit 26. Bit 10 is removed from the mouth.

A 24 gauge 0.5652 mm OD blunt injector 40 having a hub 64 is connected to a fluid-containing syringe. Injector 40 protrudes distally from hub 64 a length of 3 mm, as shown in FIG. 7B. Hub 64 has a flat, elastomeric distal end encompassing injector 40. The diameter of the distal end of hub 64 is 2.5 mm. Hub 64 is transparent such that user is able to maintain a degree of visual contact with the tip of the 3 mm long injector 40 during insertion into gingival access 22. The distal portion of hub 64 is angled 55° with respect to the long axis of the syringe to facilitate convenient access in the confined site.

Injector 40 is readily inserted into gingival access 22. The 55° angle of hub 64 with respect to the long axis of the syringe also prevents interference from the syringe, the proximal portion of hub 64, the user's hand, the patient's cheeks, and so on. The transparent, 2.5 mm diameter distal end of hub 64 does not substantially obstruct users visual contact with injector 40 when locating and inserting injector 40 into gingival access 22. User is able to maintain at least partial visual contact through the distal end while inserting injector 40 into gingival access 22. Further, the 0.5652 mm OD of injector 40 is sufficiently small relative to the 0.908 mm diameter of gingival access seat 22 such that injector 40 does impinge on slumping gingiva 24. As such, gingival access 22 is readily accessible during insertion of injector 40.

Injector 40 is readily inserted into access seat 36. The 0.5652 mm OD of injector 40 is sufficiently small relative to readily insert into the larger 0.908 mm diameter access seat 36. Injector 40 is inserted into access seat 36 until the flat distal end of hub 64 comes to rest against the surface of gingiva 24 that surrounds gingival access 22.

A substantial space is present between the 0.5652 mm diameter injector 40 and the 0.908 mm diameter gingival access 22 and access seat 36. As such, injector 40 does not sealingly engage gingival 24 or access seat 36. Therefore user increases insertional pressure on the syringe such that the flat, elastomeric distal end of hub 64 is pressed against gingiva 24 about gingival access 22. The 2.5 mm diameter of the distal end of hub 64 is sufficiently large relative to the 0.9081 mm diameter of gingival access 22 so that hub 64 does not enter gingival access 22. The elastomeric distal end of hub 64 elastically adapts to the irregular gingiva 24 surface. The elastomeric distal end of hub 64 thereby compresses gingiva 24 to form a fluid-tight seal between gingiva 24 and hub 64. Such a seal substantially prevents fluid from leaking from between hub 64 and gingiva 24.

A thickness of gingiva 24 is interposed between hub 64 and proximal bone 32. Gingiva 24 thereby shims hub 64 such that the 3 mm long injector 40 does not interferingly contact the floor of 3 mm deep access seat 36. As such, injector 40 does not interfere with hub 64 sealingly pressing against gingiva 24.

Injector 40 prevents hub 64 from inadvertently being moved away from gingival access 22. Injector 40 bumps against the sidewalls of gingival access 22 if hub 64 is inadvertently moved laterally in a direction away from gingival access 22. As such, inserted injector 40 functions to stabilize the location of hub 64 over gingival access 22.

User activates the syringe so that pressurized fluid is forced into injector 40. Pressurized fluid is ejected from injector 40 and into the space about injector 40 in access seat 36. The leak-tight seal between the distal end of hub 64 and gingiva 24 substantially prevents fluid from leaking out into the mouth. As such, the pressurized fluid from injector 40 is directed through conduit 26 and into cancellous bone 30. After sufficient volume of fluid has been injected into cancellous bone 30, the syringe with injector 40 is withdrawn from the mouth. The intraosseous injection is complete.

If reinjection is required at a later time, injector 40 is reinserted into gingival access 22 and access seat 36, the distal end of hub 64 is sealingly pressed against gingiva 24. Injected fluid is sealingly contained and directed through conduit 26 and into cancellous bone 30. The syringe with injector 40 is withdrawn from the mouth, completing the procedure.

### SUMMARY, RAMIFICATIONS AND SCOPE

Accordingly, the reader will see that the bit 10 of this invention is able to rapidly perforate cortical bone 28 and rapidly form an access seat 36 in proximal bone 32.

The process offers the advantage that an injector 40 may be rapidly sealingly engaged with proximal bone 32 or with gingiva 24 for injection through cortical bone 28 and into cancellous bone 30.

Furthermore, bit 10 and method have the additional advantage of minimizing the risk of sharps inadvertently left in the mouth.

In various alternative embodiments, the apparatus may further include configurations of countersinks 14 capable of cutting access seats 36 having undercuts to more securely, or more sealingly, retain injector 40. Other countersink 14 and access seat 36 features may be preferred for facilitating insertion or sealing of injector 40, such as slots, grooves, threads, ridges, and so on. Further, bit 10 may be formed unitarily from a single piece of metal that includes the perforator 12, countersink 14, shank 16, and even sleeve 42.

Bit 10 parts have generally been described as having dimensions that correspond to standardized needle sizes. However, OD, ID, and wall thickness sizes may be customized, and not match standard needle sizes.

Although the description above contains many specificities, these should not be construed as limiting the scope of the invention and process, but as merely providing illustrations of some of the presently preferred embodiments of this invention.

For example, countersink 14 may have a connected circular flange having a larger diameter than countersink 14, and located a few millimeters from the distal tip of countersink 14. The flange can limit the boring depth of access seat 36. The flange may or may not enlarge gingival access 22.

As a second example, a first bit comprising a perforator may be provided for forming conduit 26 and a second bit comprising a countersink may be provided for forming access seat 36. As such, both the first and second bits would be used to prepare proximal bone 32 for an injector 40. The countersink bit would preferably have a centering pin extending from the distal tip for inserting into conduit 26 while gingival access 22 or access seat 36 are formed.

As a third example, bit 10 may be used for extraoral intraosseous injections. Thus the scope of the invention should be determined by the appended claims and their legal equivalents, rather than by the examples given.

As a fourth example, countersink 14 may be removably connected to perforator 12 or shank 16. Removing countersink 14 from the remainder of bit 10 exposes a greater length of perforator 12. The additional length of perforator 12 may be used to perforate an unusually thick cortical bone 28, or to reopen a clogged conduit 26.

I claim:

1. A method for injecting fluid through a given thickness of a cortical bone having a cover of gingiva, wherein said thickness comprises a proximal bone and a distal bone, comprising the steps of: boring a conduit through said gingiva and through said thickness, sealing an injector to said gingiva or to said proximal bone wherein said injector is in fluid communication with said conduit, and injecting a fluid through at least said distal bone via said conduit.

2. The method of claim 1, wherein a sleeve is sealingly interposed between said injector and said gingiva or said proximal bone.

3. The method of claim 1, wherein a countersink is sealingly interposed between said injector and said gingiva or said proximal bone.

4. A bone bit comprising a cortical bone perforator for boring a conduit through a proximal cortical bone, through a distal cortical bone, and through gingiva covering said proximal cortical bone, and a concentric countersink for boring an enlarged conduit access at least within said gingiva, wherein said perforator has a smaller diameter than said countersink and extends distally from said countersink.

5. The bone bit of claim 4, wherein said conduit access comprises an enlarged conduit access formed within said proximal cortical bone.

6. The bone bit of claim 4, wherein a sleeve is removable from said bit for maintaining patency of said conduit access within said gingiva.

7. The bone bit of claim 6, wherein said sleeve cuts and enlarges said conduit access within said gingiva.

8. The bone bit of claim 6, wherein said sleeve is sealingly interposable between and injector and said gingiva or said proximal bone.

9. A bone bit comprising a cortical bone perforator for boring a conduit through a proximal cortical bone, through a distal cortical bone, and through gingiva covering said proximal cortical bone, and a concentric countersink for boring an enlarged conduit access within said gingiva and within said proximal cortical bone, wherein said perforator has a smaller diameter than said countersink and extends distally from said countersink.

10. The bone bit of claim 9, wherein a sleeve is removable from said bit for maintaining patency of said conduit access within said gingiva.

11. The bone bit of claim 10, wherein said sleeve cuts and enlarges said conduit access within said gingiva.

12. The bone bit of claim 10, wherein said sleeve is sealingly interposable between and injector and said gingiva or said proximal bone.
